(19) United States
(12) Patent Application Publication
(10) Pub. No.: US 2022/0213422 A1
(43) Pub. Date: Jul. 7, 2022

(54) METHODS AND SYSTEMS FOR INTRACELLULAR DELIVERY AND PRODUCTS THEREOF

(71) Applicant: CELLFE, INC., Alameda, CA (US)

(72) Inventors: Alla ZAMARAYEVA, Berkeley, CA (US); Alexander ALEXEEV, Atlanta, GA (US); Todd SULCHEK, Atlanta, GA (US); Miguel CALERO-GARCIA, San Francisco, CA (US); Ian SICHER, Alameda, CA (US); Jocelyn LOO, San Francisco, CA (US); Sewoon HAN, Albany, CA (US)

(73) Assignee: CELLFE, INC., Alameda, CA (US)

(21) Appl. No.: 17/611,454

(22) PCT Filed: May 15, 2020
(86) PCT No.: PCT/US2020/033298

§ 371 Date: Nov. 15, 2021

Related U.S. Application Data
(60) Provisional application No. 62/848,308, filed on May 15, 2019.
(60) Provisional application No. 63/023,170, filed on May 11, 2020.

(51) Int. Cl. C12M 3/06; C12M 1/42; C12N 15/90

(52) U.S. Cl. CPC C12M 23/16; A61K 48/00; C12N 15/90; C12M 35/04

(57) ABSTRACT

The present disclosure provides methods and systems for cell processing, including delivery of substances into cells. The methods and systems may comprise the use of a microfluidic device. The microfluidic device may comprise a channel comprising a compressive element. The compressive element may be configured to reduce a volume of the cell and facilitate the formation of one or more transient pores in a cell membrane of the cell. The one or more pores may permit one or more substances such as therapeutic or gene-editing reagents to enter the cell. Also provided are modified cells produced using the disclosed methods and systems.

### CROSS-REFERENCE TO RELATED APPLICATIONS

This application claims the benefit of U.S. Provisional Application No. 62/848,308, filed on May 15, 2019, and U.S. Provisional Application No. 63/023,170, filed on May 11, 2020, the disclosures each of which are incorporated herein by reference in their entireties.

### BACKGROUND

Intracellular delivery may be important for many different applications, such as gene transfection, editing, cell labeling, and cell interrogation. Conventional delivery methods, such as microinjection, electroporation, and sonoporation, have several disadvantages such as low delivery efficiencies, limited throughput, and low cell viability. Furthermore, conventional methods are generally not suitable for delivery into cell nucleus. Therefore, there is a need for new methods and systems for intracellular delivery which can overcome deficiencies in conventional techniques.

### SUMMARY

Provided herein are methods and systems for intracellular delivery. The methods and systems may include the use of a microfluidic device for delivery of various substances (such as gene-modifying reagents) to therapeutic cells. The methods and systems of the present disclosure may facilitate a process which may permeabilize cell membranes. The process may mechanically permeabilize cell membranes. The process may include rapid compression of cells to open one or more membrane pores. The one or more membrane pores may be transient pores. The one or more membrane pores may permit various substances to enter into cells. The compression may be rapid. The compression may occur in a short time period. The compression may reduce a volume of cells. After the compression and volume reduction, the cells may recover the volume by absorbing media surrounding the cells. The surrounding media may comprise one or more reagents that may be introduced into the cells as a part of the recovery process.

Accordingly, in one aspect, the disclosure provides methods for delivering a substance into a cell, comprising:

(a) providing a microfluidic device, wherein the microfluidic device comprises a channel that comprises a compressive element; and a fluid within the microfluidic device, wherein the fluid comprises the cell and the substance; and

(b) subjecting the fluid to flow through the channel in contact with the compressive element, wherein the contact causes formation of at least one pore in a membrane of the cell, wherein the at least one pore enables an entry of the substance into the cell.

In some embodiments, entry of the substance into the cell is at an efficiency greater than or equal to about 50%, the substance has an average molecular weight greater than or equal to about 1 megadaltons, and/or the cell is a vertebrate blood cell, for example a peripheral blood mononuclear cell, more specifically a lymphocyte, even more specifically a B cell, a T cell, a natural killer cell, a natural killer T cell, or a gamma delta T cell, and yet even more specifically a T cell such as a CD4+ cell or a CD8+ cell. In some embodiments, the cell is a CD34+ cell.

In some embodiments, the substance is a nucleic acid, more specifically a double-stranded deoxyribonucleic acid, a ribonucleic acid, or a nucleic acid encoding a chimeric antigen receptor.

In some embodiments, the substance is a gene editing reagent, specifically a gene editing reagent targeting a T cell receptor gene.

In some embodiments, a gap between the compressive element and an interior surface of the channel is between about 3 μm and about 15 μm, the cell has a cell diameter, and wherein a gap between the compressive element and an interior surface of the channel is less than or equal to about 20% of the cell diameter, or the compressive element is a ridge, more specifically a ridge having a width of between 15 μm and 250 μm.

In some embodiments, the cell flows through the channel at an average flow rate of from 10 mm/s to 2000 mm/s, the cell flows through the channel at an average flow rate of at least 800 mm/s, the cell flows through the channel at a rate of at least 10⁷ cells/hour, or the fluid flows through the channel at a rate of at least 400 μL/min, specifically wherein the fluid comprises a population of cells, and wherein the substance enters at least 50% of the population of cells.

In some embodiments, the cell has a volume, and the compressive element is configured to reduce the volume of the cell, specifically wherein the volume is reduced temporarily.

In some embodiments, the fluid further comprises a nanoparticle tracker, specifically an iron oxide nanoparticle.

In some embodiments, the method further comprises the step of selecting the cell for a biophysical property prior to subjecting the fluid to flow through the channel in contact with the compressive element, specifically wherein the biophysical property distinguishes CD4+ cells from CD8+ cells, is size, or is presence of a specific surface antigen.

In any of these embodiments, the channel can be defined by at least a first wall and a second wall, wherein the first wall and the second wall are substantially rigid, and more specifically wherein the channel may not comprise a diversion channel. In some of these embodiments, the first wall comprises a flexible material and a bracing material, and wherein the bracing material is positioned on an exterior surface of the first wall, more specifically wherein the bracing material is a rigid glass or plastic material. In some of these embodiments, the first wall or the second wall is prepared by injection molding, more specifically wherein the first wall or the second wall comprise a glass, a thermoplastic, or a thermosetting polymer.

In any of these embodiments, the channel may not comprise a diversion channel. More specifically in these embodiments, the channel may be defined by at least a first wall and a second wall, wherein the first wall and the second wall are substantially rigid, even more specifically wherein the first wall comprises a flexible material and a bracing material, and wherein the bracing material is positioned on an exterior surface of the first wall, yet more specifically wherein the bracing material is a rigid glass or plastic material, or wherein the first wall or the second wall is prepared by injection molding, and specifically wherein the first wall or the second wall comprise a glass, a thermoplastic, or a thermosetting polymer.

In another aspect is provided a microfluidic device comprising:

a first wall comprising a first surface, wherein the first wall extends along a direction of fluid flow;

a second wall comprising a second surface, wherein the second wall extends along the direction of fluid flow; and

a plurality of ridges connected to the first wall, wherein the plurality of ridges extends from the first surface toward the second surface, and wherein a ridge of the plurality of ridges comprises a ridge surface that forms a gap with the second surface;

wherein the first wall and the second wall are substantially rigid.

In some embodiments, the first wall comprises a flexible material and a bracing material, and wherein the bracing material is positioned on an exterior surface of the first wall, more specifically wherein the bracing material is a rigid glass or plastic material, or the first wall or the second wall is prepared by injection molding, more specifically wherein the first wall or the second wall comprise a glass, a thermoplastic, or a thermosetting polymer.

In some embodiments, a gap between the compressive element and an interior surface of the channel is between about 3 μm and about 15 μm.

In some embodiments, the compressive element is a ridge, and more specifically the ridge has a width of between 15 μm and 250 μm.

In any of these embodiments, the channel may not comprise a diversion channel.

In yet another aspect is provided a microfluidic device comprising:

a first wall comprising a first surface, wherein the first wall extends along a direction of fluid flow;

a second wall comprising a second surface, wherein the second wall extends along the direction of fluid flow; and

a plurality of ridges connected to the first wall, wherein the plurality of ridges extends from the first surface toward the second surface, and wherein a ridge of the plurality of ridges comprises a ridge surface that forms a gap with the second surface;

wherein the channel does not comprise a diversion channel.

In some embodiments, a gap between the compressive element and an interior surface of the channel is between about 3 μm and about 15 μm, or the compressive element is a ridge, more specifically wherein the ridge has a width of between 15 μm and 250 μm.

In any of these embodiments, the first wall and the second wall may be substantially rigid, more specifically wherein the first wall comprises a flexible material and a bracing material, and wherein the bracing material is positioned on an exterior surface of the first wall, even more specifically wherein the bracing material is a rigid glass or plastic material, or wherein the first wall or the second wall is prepared by injection molding, more specifically wherein the first wall or the second wall comprise a glass, a thermoplastic, or a thermosetting polymer.

In still yet another aspect is provided a modified cell prepared by any of the above method embodiments.

In some embodiments, the cell retains high proliferative capacity, more specifically the cell is a T cell, even more specifically the T cell retains high cytotoxic potential and/or the T cell proliferates within 10 days of delivery of the substance into the cell. In other more specific embodiments, the cell is a CD34+ cell, even more specifically the cell proliferates within 24 hours of delivery of the substance into the cell.

In some embodiments, the at least one pore is a transient pore and/or the at .least one pore is no longer present in the membrane.

In some embodiments, the modified cell is substantially free of a transfection agent, more specifically wherein the transfection agent is a chemical transfection agent or wherein the transfection agent is a biological transfection agent.

In any of these embodiments, the modified cell can be obtained with a yield of at least 20%.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1A shows a schematic cross-sectional view of an example microfluidic device of the present disclosure.

FIG. 1B shows a schematic cross-sectional view of an example microfluidic device of the present disclosure.

FIG. 2 shows an example of plasmid transfection in cancer cell line JX-17 in a microchannel with chevron ridges and 7.6 micrometer gap size.

FIGS. 3A-3E show example designs of microfluidic device comprising microchannels. FIG. 3A shows examples of microchannel layout with chevron ridges with and without cell focusing element. FIG. 3B shows an example of cell focusing through Dean's flow design without the need for sheath flow focusing. FIG. 3C shows an example device with 5 parallel microchannels having chevron ridges and Dean focusing. FIG. 3D shows an alternative design with 4 separate microchannels, no cell focusing element, fewer chevron ridges, and ridges located near the end of the microchannel. FIG. 3E shows exemplary highly parallel microchannel designs suitable for scaling up rates of cell processing. Flow is from left to right in each case.

FIGS. 4A-4E show the deformation of a non-rigid channel device as the fluid flow rate through the channel increases. FIG. 4F shows the lack of deformation in a channel design having a glass brace backing the ridge structure.

FIGS. 5A-5B show top-down and two side cross-sectional views of a microfluidic device with a glass-braced PDMS ridge, either with no fluid flow (FIG. 5A) or with 800 μL/min fluid flow (FIG. 5B).

FIGS. 6A-6D show the effect of fluid flow rates on cell viability, cell recovery, cell transfection, and total transfection yield in CD4+ and CD8+ cells processed using a microfluidic device with a glass-braced PDMS ridges.

FIGS. 7A-7D show the increased transfection rates of cells processed using microfluidic devices with microchannels lacking diversion channels.

FIGS. 8A-8B show the transfection of CD4+ and CD8+ cells with CRISPR/Cas9 gene editing reagents to knock out native T cell receptor functionality.

FIG. 9 shows the transfection of peripheral blood mononuclear cells with mRNA.

FIG. 10 shows a comparison of transfections of unactivated (“naïve”) and activated T cells using two different microfluidic devices (small gap and large gap).

FIG. 11A. Transfection results for CD4+ and CD8+ T cells with GFP mRNA. Left, transfection efficiency as percentage of live cells that becomes GFP positive. Right, recovery and viability relative to no device (negative) control. FIG. 11B. Transfection results for CD4+ and CD8+ T cells with TRAC CRISPR/Cas9 RNP. Left, TCR KO efficiency as percentage of live cells that cannot be labelled with TCRαβ antibody. Right, recovery and viability relative to no device (negative) control.

FIG. 12A. Relative T cell expansion after transfection with a volume exchange for convective transfer (VECT) device, or no device (negative) control condition. FIG. 12B. Analysis of exhaustion markers in CD4+ and CD8+ cells 7 days after transfection with VECT, or in the no device (negative) control. P, PD-1; T, TIM-3: L, LAG-3: C, CTLA-4.

FIG. 13A. Viability and recovery of PBMCs at different cell concentrations for VECT. FIG. 13B. Viability and recovery of PBMCs at different flow rates for VECT.

FIG. 14. Lymphocyte panel allowing identification of various lymphocytes.

FIG. 15. Naive PBMCs flowed through a subject device at a constant flow rate. 24 hours later, the PBMCs were analyzed with a lymphocyte panel designed to be read out on the flow cytometer. The results demonstrate that the device can successfully deliver mRNA into different lymphocytes, including two cell types of interest: NK and γδ T cells.

FIG. 16. NK cells were isolated from PBMCs and then flowed through various devices at a constant flow rate. Flow was performed with isolated NK cells to identify which gap size was the most ideal for this cell type.

FIG. 17A. Exemplary VECT device. Cells mixed with payload are used as input, run through a microfluidic channel, and the final engineered product is collected afterwards. FIG. 17B. A top-down microscopic view of the entrance of the microfluidic channel. Dotted line indicates where the equivalent cross-section of the channel is drawn in FIG. 17C. Dotted square of FIG. 17C is further magnified in FIG. 17D for a diagram of how serial compressions work.

FIGS. 18A-18E. GFP mRNA transfection was achieved with VECT and compared to a commercial electroporator 48 hours after processing. FIG. 18A. Transfection efficiency as the percentage of live cells that become GFP positive. Viability of the cells (FIG. 18B) and cell recovery of live cells (FIG. 18C). FIG. 18D. Product yield is calculated by multiplying transfection efficiency and recovery of live cells, in order to understand what is the percentage of cells engineered from the total number of cells input in the system. FIG. 18E Normalized cell proliferation after cells are transfected with mRNA.

The patent or application file contains at least one drawing executed in color. Copies of this patent or patent application publication with color drawing(s) will be provided by the Office upon request and payment of the necessary fee.

### DETAILED DESCRIPTION

Ex vivo genetic modification of therapeutic cells may hold the promise of providing a lifelong cure for diseases. For example, gene therapy of hematopoietic stem and progenitor cell (HSPC) may be a route to treat blood diseases such as sickle cell disease, beta thalassemia, adenosine deaminase deficiency severe combined immune deficiency (ADA-SCID), HIV, and others. Unlike allogeneic bone marrow transplant, these autologous approaches generally do not suffer from risks of graft versus host disease (GVHD) and may be less prone to graft failure. Autologous treatments may not require a human leukocyte antigen (HLA)-matched marrow donor, but in practice access to stem cell gene therapy still remains limited. In addition, HSPC gene therapy still suffers from significant drawbacks. A main bottleneck may be the cost of cell product development and manufacturing. For example, HSPCs may need to be harvested, isolated, and cultured before administration of a complex and costly series of genetic manipulations, all in a good manufacturing practice (GMP) environment before formulation and administration several weeks after harvest. These challenges can render HSPC gene therapies expensive and arduous to develop.

The core of gene modification in cell therapy may be delivery of therapeutic transgenes to patient cells. Gene-modified stem cell therapies can primarily rely on either viral transgene delivery, electroporation, or both. Viral gene therapy (using modified retrovirus or, more recently, lentivirus as vector) may have a longer track record. Nonetheless viral gene therapy may carry significant drawbacks, including the high cost, complexity, and variable quality of vector manufacturing (costing upward of 100,000 dollars per dose), and the risk of insertional mutagenesis from randomly integrating vectors. These factors may make vector manufacturing a prominent limitation to viral gene therapy in HSPCs. As an alternative, non-viral gene therapies, many relying on genome editing, may have been a recent focus of development.

Despite early successes in clinical trials, ex vivo genomic modification remains cumbersome and expensive. In ex vivo gene therapy (or gene editing), cells may be harvested from affected patients, genetically modified, formulated, and re-infused into the patient. A major hurdle in this process may include the delivery of gene-modifying reagents to the cells. Viral delivery can be expensive and unreliable, hindered by inefficiencies and variability associated with diffusion. Moreover, large therapeutic transgenes (greater than 10 kB), may be difficult to package into viral genomes. Conventional methods such as electroporation may have demonstrated certain level of successful transfections. However, these conventional methods have several disadvantages such as low transfection efficiency, low cell viability, and high off-target variations of the gene expression of cells after electrical shock. In addition, for the conventional methods, it may be challenging to scale from bench top to clinical production. For example, significant and costly delays may result from scaling up research electroporation (1M cells) procedures to patient scale (>100M cells), which procedures may include: a switch of instrument suppliers, re-optimization of the protocol from scratch using the new instrument, and scaling up in increments. Therefore, there is a need for new delivery methods and systems to serve the cell therapy space, and ultimately to expand access to various life-changing therapies.

Methods and systems for intracellular delivery can generally be divided into the following non-limiting classes: a) physical/non-viral approaches, such as mechanoporation, gene gun, ultrasound, electroporation, and laser; b) chemical/non-viral approaches, such as cationic lipids/liposomes, polysaccharides, cationic polymers, cationic peptides, and micro-/nano-particles; and c) biological/viral approaches.

Among the physical/non-viral approaches, electroporation is most commonly used to transfect nucleic acids into higher cells. Although electroporation can, in principle, be applied to all cell types and at all stages of the cell cycle, damage to a cell by electroporation can be serious, compared with some other physical methods. Although the principle of electroporation is applicable to all cell types, its efficiency can depend on the electrical properties of the cells. Smaller cells require higher electrical fields to permeate. This is an important consideration for ex vivo gene delivery, especially to hematopoietic cells. Cells with less conductive contents (such as adipocytes) are considered to be less susceptible to damage from electroporation. For charged substances, such as nucleic acids, electroporation is often improved by including non-natural chemical agents in the formulations. Finally, electroporation requires the use of conductive buffers, and it is not suitable for the intracellular delivery of metallic substances, such as, for example, nanoparticle trackers comprising iron oxide nanoparticles.

Provided herein are methods and systems for intracellular delivery, as well as cellular products of the disclosed methods and systems. The methods and systems facilitate delivery of one or more substances (such as therapeutic reagents or gene-editing reagents) into cells. The methods and systems include the use of a microfluidic device. The microfluidic device comprises a channel which comprises a compression element. Without intending to be bound by theory, it is believed that the compression element facilitates a process in which cell membranes are permeabilized. As a cell flows through the microfluidic device, the cell comes into contact with the compression element within the channel. This contact is believed to result in a cell volume reduction. After the compression, the cell may recover part or all of its reduced volume by absorbing its surrounding media. The surrounding media may include one or more substances which may be transported into the cell during the recovery process.

The one or more substances delivered to cells according to the above methods and systems may comprise a plurality of substances. The plurality of substances may be large molecules. The plurality of substances may have an average molecular weight greater than or equal to about 0.5 megadaltons (MDa), 0.6 MDa, 0.7 MDa, 0.8 MDa, 0.9 MDa, 1.0 MDa, 1.1 MDa, 1.2 MDa, 1.3 MDa, 1.4 MDa, 1.5 MDa, 1.6 MDa, 1.7 MDa, 1.8 MDa, 1.9 MDa, 2.0 MDa, 2.1 MDa, 2.2 MDa, 2.3 MDa, 2.4 MDa, 2.5 MDa, 2.6 MDa, 2.7 MDa, 2.8 MDa, 2.9 MDa, 3.0 MDa, 3.5 MDa, 4.0 MDa, 4.5 MDa, 5.0 MDa, or more. In some cases, each of the substances may has a molecular weight that is greater than or equal to about 0.5 megadaltons (MDa), 0.6 MDa, 0.7 MDa, 0.8 MDa, 0.9 MDa, 1.0 MDa, 1.1 MDa, 1.2 MDa, 1.3 MDa, 1.4 MDa, 1.5 MDa, 1.6 MDa, 1.7 MDa, 1.8 MDa, 1.9 MDa, 2.0 MDa, 2.1 MDa, 2.2 MDa, 2.3 MDa, 2.4 MDa, 2.5 MDa, 2.6 MDa, 2.7 MDa, 2.8 MDa, 2.9 MDa, 3.0 MDa, 3.5 MDa, 4.0 MDa, 4.5 MDa, 5.0 MDa, or more.

The substances may or may not comprise a charged substance. The substances may comprise a drug, a nucleic acid molecule, an antigen, a polypeptide, an antibody, an antigen, a hapten, an enzyme, or combinations thereof. The nucleic acid molecule may comprise deoxyribonucleic acid (DNA), ribonucleic acid (RNA), peptide nucleic acid (PNA), or combinations thereof. The substances may be modified using e.g., nuclear locators. The nuclear locators may comprise nuclear localization signal (NLS) locators.

The method may further comprise subjecting one or more cells to flow through the channel of the microfluidic device. As the cell or cells flow through the channel, the cell or cells may be in contact with the compression element which is comprised in the channel. The channel may have a cross-sectional dimension that is greater than or equal to about 1 micrometers (μm), 5 μm, 10 μm, 15 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, 500 μm, 600 μm, 700 μm, 800 μm, 900 μm, 1,000 μm, or more. In some cases, the cross-sectional dimension of the channel may be less than or equal to about 2,000 μm, 1,500 μm, 1,000 μm, 850 μm, 700 μm, 550 μm, 400 μm, 300 μm, 200 μm, 100 μm, 80 μm, 60 μm, 40 μm, 20 μm, 10 μm, or less. In some cases, the cross-sectional dimension of the channel may fall within any of the two values described above, e.g., between about 20 μm and about 1,000 μm, or between about 50 μm and about 100 μm.

The cell or cells may be any types of cells. Non-limiting examples of cells may include plant cells, animal cell, human cells, insect-derived cells, bacteria, adherent cells, suspension cells, cardiomyocytes, primary neurons, HeLa cells, stem cells, ESCs, iPSCs, hepatocytes, primary heart valve cells, gastrointestinal cells, k562s, lymphocytes, T-cells, Bcells, natural killer cells, dendritic cells, hematopoetic cells, beta cells, somatic cells, germ cells, embryos (human and animal), zygotes, gametes, 1205 Lu, 1321N1, 143B, 22Rv1, 23132/87, 293, 293 (suspension), 293-F, 293T, 2A8, 2PK3, 300.19, 32D, 3A9, 3T3-L1 ad, 3T3-L1 pre-ad, 3T3-Swiss albino, 4T1, 5838 Ewing's, 661W, 697, 7-17, 720, 721.174, 721.22, 721.221, 786-0, A-10, A-375, A-431, A-498, A-673, A172, A2.A2, A20, A2058, A2780, A3.01, A549, A7r5, Adipocyte (pre), Adipocyte (pre)-human diabetes Tp.2, Adipose stem cell-human diabetes Tp.1, Adipose stem cell-human diabetes Tp.2, Adipose stem cell, Adrenocortical, AGN2a, AGS, AML, AML-DC, ARH 77, ARPE-19, arteries mesenteric (MA), astrocyte glioblastoma line-mouse, Astrocyte-human (NHA), Astrocyte-mouse, Astrocyte-rat, Astrocyte, ASZ001, AT-1, ATDC5, B cell-human, B-cell-lymphoma cell line, B-cell-mouse-stimulated, B-CLL, b-END, B157, B16-F0, B16-F1, B16-F10, B35, B3Z, B65, BA/F3, Babesia bovis, Balb/c 3T3, BC-1, BCBL1, BCL1 clone 5B1b, BCL1.3B3, BE2-M17, BEAS-2B, Beta islet cell, BeWo, BHK-21, BHP2-7, BJ, BJ1-hTERT, BJAB, BJMC3879, BL2, BL3, BLCL, BPH1, BRIN-BD11, BT-20, BT549, BV173, BV2, BW5147, BW5147.3, BxPC-3, C10/MJ2, C17.2, C28A2, C2C12, C2F3, C3H10T1/2, C57MG, C6, C8161, CA46, Caco-2, Caco-2/TC7, Cal-1, Cal-85-1, CAL51, Calu-3, Calu-6, CAMA 1, CAP (CEVEC's Amniocyte Production), Capan-1, Capan2, Cardiomyocyte, CCD18Co, CCRF-CEM, CCRF-CEM C7, CD34+ cell, CEM-C7A, CEM.C1, Cervical stroma, CFBE, CH1, CH12, CH12F3, CH27, CHM 2100, CHO (suspension), CHO-DG44, CHO-DG44 (DHFR-), CHO-K1, CHO-S cells sold under the trademark FREESTYLE by Thermo Fisher Scientific (Waltham, Mass.), CHO-S (suspension), Chondrocyte (human (NHAC-kn)), Chondrocytes (mouse), Chromaffin cells (cow), CML, Colo201, Colo205, Colo357, Cor.At Cardiomyocytes (from ESC-mouse), COS-1, COS-7, CRFK, CTLL-2, CV1, Cytokine induced killer, Cytotrophoblast, D1 ORL UVA, D1F4, D283, D425, D54, Dante-BL, Daudi, DCIS, Dendritic cell (human), Dendritic cell (mouse-immat.-BALB/c), Dendritic cell (mouse-immat.-C57BL/6), Dendritic cell (mouse-mature-BALB/c), Dendritic cell (mouse-mature-C57BL/6), Dendritic cell (plasmacytoid-human), Dendritic cell (rhesus macaque), DEV, DHL4, DHL6, DLD-1, DO11.10, DOHH-2, Dorsal root gang (DRG), Dorsal root gang (DRG) (rat), Dorsal root gang (DRG) (chicken), Dorsal root gang (DRG) (mouse), DOV13, DPK, DT40, DU 145, EAhy926, eCAS, ECC-1, EcR293, ECV304, Eimeria tenella, EJM, EL4, Embryonic fibroblast, Embryonic fibroblast (chicken), Embryonic fibroblast (mouse (MEF) immort), Embryonic fibroblast (mouse (MEF) primary), Embryonic stem (ES) cell (human), Embryonic stem (ES) cell (mouse), EMC, Endothelial, Endothelial-aortic-cow (bAEC), Endothelial-aortic-human (HAEC), Endothelial-aortic-pig, Endothelial-coronary art-human (HCAEC), Endothelial-lung-sheep, Endothelial-Mammary-Human, Endothelial-MV dermal-human adult, Endothelial-MV dermal-human neo, Endothelial-MV lung-human (HMVEC-L), Endothelial-pulmonary artery-human, Endothelial-umbilical vn-human (HUVEC), EpH4, Epithelial, Epithelial model-cornea-human-immort, Epithelial-airway-human, Epithelial-airway-pig, Epithelial-alveolar-rat, Epithelial-bronchial (NHBE)-human, Epithelial-bronchial-monkey, Epithelial-cornea-human, epithelial-ES-derived-human, Epithelial-lung type II-human, Epithelial-mammary-human (HMEC), Epithelial-mammary-mouse, Epithelial-prostate (PrEC)-human, Epithelial-renalhuman (HRE), Epithelial-retinal pigment-human, Epithelial-Small Airway-human (SAEC), ESS-1, F36P, F9, FaO, FDC-P1, FDCP-Mix, Fibroblast, Fibroblast-aortic adventitial-human, Fibroblast-cardiac-rat, Fibroblast-cow, Fibroblast-dermal (NHDF-Neo)-human neo, Fibroblast-dermal (NHDF-Ad)-human adult, Fibroblast-dermal-human, Fibroblast-dermalmacaque, Fibroblast-ES-derived-human, Fibroblast-foreskin-human, Fibroblast-humanGM06940, Fibroblast-lung-human normal (NHLF), Fibroblast-lung-mouse, Fibroblast-lungrat, Fibroblast-pig, Fibroblast-tunica albuginea-human, FL5.12A, FM3A, FRT, G-361, GaMG, GD25, GH3, GIST882, GM00131, GM05849, GM09582, Granta519, Granule cell, Granule cell (CGC)-mouse, Granule cell (CGC)-rat, GT1-7, H2K mdx, H4, H4IIE, H69, H9, H9c2(2-1), HaCaT, HC11, HCA7, HCC1937, HCC1954, HCT 116, HCT15, HDLM-2, HDQ-P1, HEK-293, HEL 92.1.7, HeLa, HeLa S3, Hep G2, Hep1B, HEPA 1-6, Hepa-1c1c7, Hepatocyte, Hepatocyte immortalized-mouse, Hepatocyte-human, Hepatocyte-mouse, Hepatocyte-rat, HFF-immort, HFF-1, HFFF2, HIB1B, High Five, HK-2, HL-1, HL-60, HMC-1, HMEC-1, HMLE, HMy2.CIR (C1R), HN5, HPB-ALL, Hs 181.Tes, Hs 578T, HT-1080, HT-29, HT22, HT29-D4, HTC, HU609, HuH7, HuT 102, HuT 78, HUV-EC-C, IEC-6, IEC18, IGROV1, IHH, IM9, IMR-32, IMR-90, INS-1, INS-1E, INS1 832/13, IOSE29, IOSE80, iPS-human, J-774, J-Lat 6.2, J558L, J774A.1, JB6-1, JB6-2, JeKo-1, Jurkat, Jurkat-modified, JVM, JVM-2, K-562, Karpas 299, KE-37, Kelly, Keratinocyte, Keratinocyte-(NHEK-Ad) human adult, Keratinocyte-(NHEK-neo) human neonatal, KG-1, KG-1a, KHYG1, KIT225, KM-H2, KS, KTA2, Ku812, L-428, L1.2, L1210, L1236, L3.6SL, L5178Y, L540, L6, L87/4, LA-N-2, LA-N-5, LAMA-84, Langerhans cells, Langerhans cells-human, LAZ 221, LbetaT2, LCL, Leishmania tarentolae, LLC-MK2, LLC-PK1, LLC-PK10, LN229, LNC, LNCaP, LoVo, LP1, LS180, LX-2, LY2, M-07e, M28, MA 104, Macrophage, Macrophage-human, Macrophage-mouse, Macrophage-mouse-BALB/c, Macrophage-mouse-C57BL/6, MC-38, MC/9, MC3, MC3T3, MC3T3-E1, MC57G, McA-RH7777, MCF10, MCF10A, MCF7, MCF7 tet, MCT, MDA-MB-231, MDA-MB-361, MDA-MB-415, MDA-MB-453, MDA-MB-468, MDBK, MDCK, MDCK II, MDCK-C7, ME-1, MedB1, MEG-01, MEL, melan-a, Melanocyte, Melanocyte-(NHEM-neo)-human neonatal, Mesangial cells-Human (NHMC), Mesench. stem (MSC)-pig, Mesenchymal stem cells, Mesenchymal stem cell (MSC)-human, Meso17, Met-1fvb2, MEWO, MFM223, MG-63, MGR3, MHP36, MiaPaCa-2, mIMCD3, MIN6, Mino, MKN-1, mlEND, MLO-Y4, MLP29, MM.1S, MN9D, MOLM-14, MOLT-4, Molt16, Monocyte, MonoMac1 (MM1), MonoMac6 (MM6), Mouse L cell, MPC-11, Mpf, mpkCCD(c14), MPRO, MRC-5, MT4, MTC, MTLn3, Mutu1, MUTZ-2, MUTZ3, MV-4-11, Myoblast, Myoblast-(HSMM) human, Myofibroblast, Myofibroblast-human hepatic, Myofibroblast-rat hepatic, MzCHA-1, N11, N114P2, N1E115, N9, NALM-6, Namalwa, Natural killer (NK)-human, NB-4, NBL-6, NCEB-1, NCI-H1299 (H1299), NCI-H1435, NCI-H2170, NCI-H226 (H226), NCI-H292, NCI-H295R (H295R), NCIH358 (H-358; H358), NCI-H460 (H460), NCI-H69 (H69), NCI-H929 (H929), NCM460, NCTC clone 929, Neural precursor-cow, Neural stem cell (NSC), Neural stem cell (NSC)-human, Neural stem cell (NSC)-mouse, Neural stem cell (NSC)-rat, Neuro-2a (N2a), Neuroblastoma, Neuron-cortical-mouse, Neuron-hippo/cortical-rat, Neuron-hippocampal-chicken, Neuronhippocampal-mouse, Neuron-mesencephalic-rat, Neuron-striatal-mouse, Neuron-striatal-rat, NG108-15, NIH/3T3, NK-92, NK3.3, NKL, NKL1, NRK, NRK-49F, NRK52E, NS0, NS1, NSC34, NTERA-2 cl.D1, OCI-AML1a, OCI-AML2, OCI-AML3, OCI-LY-10, OCI-LY-3, Olfactory neuron-rat, Oligodendrocyte-rat, OP-6, OVCAR3, P. knowlesi, P19, P3X63Ag8, P815, PAC2, Pam212, PANC-1, Panc89, PBMC-human, PC-12, PC-3, Perkinsus marinus, Plasmodium berghei, Plasmodium falciparum, Plasmodium yoelii, PLB-985, PMC42, Podocytemouse, PS1, PtK1, R28, R9ab, RAEL, RAG2−/−R2BM3-7, Raji, Ramos, Rat2, RAW 264.7, RBL, RBL-1, RBL-2H3, RCC26, RD, REH, Renal Cell Carcinoma, Renal proximal tubule cells human, RF/6A, RFL-6, Rh4, Rin 1046, RIN m5f, RKO, RL-952, RMAS, RPMI8226, RS4-11, RT4, RWPE-1, S1A.TB.4.8.2, S49, SA1N, SAM-19, Saos-2, SbC12, Schneider's Drosophila Line 2, Schwannoma cell line, SCI-ET27, SCID.adh, SET-2, Sf9 (ovarian), Sf9 (ovarian), SGHPL-4, SH-SYSY, SIRC, SK-BR-3, SK-MEL 100, SK-MEL 103, SK-MEL 147, SK-MEL 173, SK-MEL 187, SK-MEL 19, SK-MEL 192, SK-MEL 197, SK-MEL 23, SK-MEL 29, SKMEL 31, SK-MEL 85, SK-MEL 94, SK-MEL-28, SK-MEL-5, SK-N-AS, SK-N-DZ, SK-N-FI, SK-N-MC, SK-N-SH, SK-OV-3, Skeletal muscle-(SkMC) human, SKNAS, SKW6.4, SMCairway (HASM)-human, SMC-aortic (AoSMC)-human, SMC-aortic (AoSMC)-mouse, SMCaortic (AoSMC)-pig, SMC-aortic (AoSMC)-rat, SMC-bladder (BdSMC)-human, SMCbronchial-human normal (BSMC), SMC-cervix-human, SMC-coronary artery-human (CASMC), SMC-coronary-rat, SMC-pul.artery (PASMC)-human, SMC-rat, SMC-ureterhuman, SMC-uterus-human (UtSMC), SMC-vascular-human, SMC-vascular-monkey, SMCvascular-rat, SP2/0, SP53, Stroco5, SUIT-2, SUM52PE, SUP-T1, SVEC 4-10, SW13, SW1353, SW48, SW480, SW620, SW837, SW872, Synoviocyte-human, SZ95, T cell line-chicken, T cell-human peripheral blood unstim., T cell-human stim., T cell-mouse-BALB/c, T cellmouse-C57BL/6, T cell-rabbit-stimulated, T-47D, T/C-28 a2, T/G HA-VSMC, T0, T1165, T2, T24, T84, TA3, TF-1, TG40, TGW, THP-1, TK6, TOM-1, Tot2, Trabecular meshwork-human, Trabecular meshwork-pig, Trophoblast-human, Trophoblast-mouse, Trypanosoma brucei, Trypanosoma congolense, Trypanosoma cruzi, TS/A, TT, Turbinate cell-cow, U-2 OS, U-2940, U-87 MG, U-937, U138MG, U251, U251MG, U266B1, U373, U373MG, U87, UACC903, UMR 106-01, UMSCC-14A, UT7, UT7 GM-CSF dependent, UT7-Epo, UT7-EpoS1, UT7-TPO, V5, V79, VAL, Vero, WEHI-231, WEHI-279, WERI-Rb-1, WI-38, WIL2-S, WM-266-4, WM35, WRO, XG6, XG6, Z-138, Zebrafish cell line, ZF4, or combinations thereof. In some cases, the cell or cells comprise T cells, hematopoietic stem cells (HSCs), induced pluripotent stem cells (iPSCs), Chinese hamster ovary (CHO) cells, or combinations thereof. In some cases, the cell or cells comprise a B cell, a T cell, a natural killer cell, a natural killer T cell, or a gamma delta T cell.

The compressive element may facilitate the formation of one or more pores in cell membranes of at least a portion of the cell or cells. For example, as the cell or cells flow through the channel, the compressive element may facilitate the formation of membrane pores in cell membrane of at least about 10%, 15%, 20%, 25%, 30%, 35%, 40%, 45%, 50%, 55%, 60%, 65%, 70%, 75%, 80%, 85%, 90%, 95% of the cells, or more. The membrane pores may be transient. The one or more membrane pores may permit at least a subset (e.g., at least about 10%, 15%, 20%, 25%, 30%, 35%, 40%, 45%, 50%, 55%, 60%, 65%, 70%, 75%, 80%, 85%, 90%, 95%) of the substances comprised in the channel to enter the cell or cells. The substances may be delivered or transported into the cell or cells with a high efficiency. The efficiency may be defined as a ratio of the cells which have substances transported therein to the total cells that pass through the channel. As an example, if 50% of the total cells have substances transported therein, then the efficiency is 50%. In some cases, the efficiency can be greater than or equal to about 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more.

As discussed above, the compression element may be configured to compress the cell or cells. The compression may be rapid. The compression may occur within a short time period. For example, the compression may occur in less than or equal to about 2 seconds (s), 1.8 s, 1.6 s, 1.4 s, 1.2 s, 1 s, 900 milliseconds (ms), 800 ms, 700 ms, 600 ms, 500 ms, 400 ms, 350 ms, 300 ms, 280 ms, 260 ms, 240 ms, 220 ms, 200 ms, 180 ms, 160 ms, 140 ms, 120 ms, 100 ms, 90 ms, 80 ms, 70 ms, 60 ms, 50 ms, 40 ms, 30 ms, 20 ms, 10 ms, 5 ms, 1 ms, or less. In some cases, the compression may occur within a time period that falls between any of the two values described above, for example, between about 10 ms and about 300 ms.

The compression may deform the cell or cells. The compression may reduce a volume of the cell or cells. The volume reduction may be temporary. The compression may cause a cell to lose at least about 10%, 15%, 20%, 25%, 30%, 35%, 40%, 45%, 50% of its volume, or more. The compressed state may be a non-natural state for the cell or cells and the cell or cells may attempt to recover to the original volume. As a result, the compression may be followed by cell expansion and recovery. During the recovery, the cell or cells may increase their volume by absorbing surrounding media which may comprise the substances, which substances may then enter the cell or cells via the one or more membrane pores.

The compression element may comprise a plurality of compressive surfaces, e.g., greater than or equal to about 2, 3, 4, 5, 6, 7, 8, 9, 10, 11, 12, 13, 14, 15, 16, 17, 18, 19, 20 compressive surfaces, or more. The compressive surfaces may be ridges. The compressive surfaces may or may not extend parallel with respect to one another. In some cases, at least a subset of the compressive surfaces extends parallel with respect to one another. The compressive surfaces may have regular or irregular cross-sectional shapes. In some cases, the compressive surfaces have rectangular cross-sections.

Dimensions of the compressive surfaces may vary, depending upon various factors, such as cell flow rate, cell type, cell size, cell stiffness, cell adhesiveness, substance type, channel material and/or channel size. For example, in some cases, the compressive surfaces have an average width that is greater than or equal to about 1 μm, 5 μm, 10 μm, 15 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, 500 μm, or more. In some cases, the compressive surfaces have an average width that is less than or equal to about 800 μm, 700 μm, 600 μm, 500 μm, 400 μm, 300 μm, 200 μm, 150 μm, 100 μm, 80 μm, 60 μm, 40 μm, 20 μm, or less. In some cases, the compressive surfaces have an average width that falls between any of the two values described above, for example, between about 20 μm and 250 μm, between about 15 μm and 250 μm, between about 10 μm and 250, between about 5 μm and 250 μm, or between about 5 μm and 100 μm.

So that the cell or cells may pass through the channel, the compressive element may have a dimension (e.g., a height) that is smaller than a cross-sectional dimension of the channel. Consequently, there may be a gap between the compressive element and an interior surface of the channel. The gap may have a size that is adjustable. The size may be a height of the gap. The gap size may be adjusted based upon a variety of factors, such as cell size, cell type, cell stiffness, cell adhesiveness, flow rate, channel material, channel size, temperature, substance type, and/or substance size. In some cases, the gap size may be greater than or equal to about 0.1 μm, 0.5 μm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 22 μm, 24 μm, 26 μm, 28 μm, 30 μm, or more. In some cases, the gap size may be less than or equal to about 50 μm, 45 μm, 40 μm, 35 μm, 30 μm, 25 μm, 20 μm, 18 μm, 16 μm, 14 μm, 12 μm, 10 μm, 8 μm, 6 μm, 4 μm, 2 μm, 1 μm, or less. In some cases, the gap size may fall within a range of any of the two values described above, for example, between about 1 μm and about 20 μm, or between about 3 μm and 15 μm.

The gap size may be smaller than a cell size. For example, the gap size may be less than or equal to about 50%, 45%, 40%, 35%, 30%, 25%, 20%, 15%, 10% of an average diameter of the cell or cells, or less. In some cases, the gap size may be less than or equal to about 50%, 45%, 40%, 35%, 30%, 25%, 20%, 15%, 10% of a diameter of a given cell comprised in the cells that pass through the channel.

In cases where multiple compressive elements (e.g., compressive surfaces) are comprised in the channel, each compressive element may have the same or a different dimension. As a result, gap sizes between each compressive element and an interior surface of the channel may or may not differ. In some cases, at least a subset (e.g., at least about 5%, 10%, 15%, 20%, 30%, 35%, 40%, 45%, 50%, or more) of the compressive elements have different dimensions.

The compressive elements may be spaced apart from one another. Such configuration may facilitate periodic compression and expansion of the cell or cells. For example, as a cell passes through the channel, the cell may be compressed while in contact with a compressive element. Following the compression and prior to being subjected to contact with a subsequent compressive element, the cell may flow into an area between the two adjacent compressive elements where the cell may expand and recover some or all of the volume lost during the compression. A space between each pair of adjacent compressive elements may or may not be the same. In some cases, the compressive elements are equally distant. In some cases, a space between each pair of adjacent compressive elements progressively increases or decreases along a flow direction of the cell or cells. The flow direction may be the main flow direction of a majority of the cells. The flow direction may be in alignment with a principal axis of the channel. The flow direction may be a direction from an inlet of the channel to an outlet of the channel.

In some cases, cells can be sticky and may tend to adhere to each other. To facilitate flow of the cell or cells within the channel and/or to maintain high flow rates, the method as provided herein may further comprise applying or forming a coating on at least a portion of an interior surface of the channel. Additionally or alternatively, a coating may be formed on at least a portion of a surface of the compressive element. The coating may be hydrophilic. In some cases, the coating comprises hydrophilic polymers.

In another aspect, methods of the present disclosure may comprise providing a microfluidic device. The microfluidic device may be a device as described above or elsewhere herein. For example, the device may comprise a channel which may comprise a compressive element and a plurality of substances. The substances can be any types of substances as described above or elsewhere herein. For example, the substances may comprise a drug, a nucleic acid molecule, an antigen, a polypeptide, an antibody, an antigen, a hapten, an enzyme, or combinations thereof. The substances may or may not comprise a charged substance. The substances may comprise therapeutic molecules or gene-editing reagents. Examples of the substances may include, but are not limited to, clustered regularly interspaced short palindromic repeats (CRISPR) associated endonuclease (Cas, such as Cas9), trans-activating RNA (tracrRNA), CRISPR-RNA (crRNA), transcription activator-like effector nucleases (TALEN), zinc finger nuclease (ZFN), guide ribonucleic acid (guide RNA), single stranded donor oligonucleotides (ssODN), messenger ribonucleic acid (mRNA), precursor mRNA (pre-mRNA), bacterial artificial chromosome (BACs), peptide nucleic acid (PNA), P-form deoxyribonucleic acid (pDNA), chromosomes, mitochondria, small interfering RNA (siRNA), short hairpin RNA (shRNA), microRNA (miRNA), proteins (such as Cas proteins including Cas9, Cpf1, C2c1, C2c3, C2c2, or combinations or modified versions thereof), morpholinos, metabolites, small molecules, peptides, antibodies, nanobodies, fluorescent tags and/or dyes, molecular beacons, deoxyribonucleic acid (DNA) origami, nanoparticles, subcellular organelles, ribozymes, enzymes, microbial pathogens, episomal vectors, or combinations thereof. In some cases, the substances comprise green fluorescent protein (GFP) DNA plasmid, GFP mRNA, Cas9, dCas, Cas9 RNP, dCas RNP, or combinations thereof.

In situations where the substance to be delivered to a cell is a polypeptide, or comprises a polypeptide, the polypeptide itself may be delivered to the cell, or a nucleic acid encoding the polypeptide may be delivered to the cell, and the polypeptide may accordingly be expressed within the cell from the nucleic acid. Likewise, where the substance to be delivered to a cell is a ribonucleic acid, or comprises a ribonucleic acid, the ribonucleic acid itself may be delivered to the cell, or a deoxyribonucleic acid encoding the ribonucleic acid may be delivered to the cell for expression there. In some situations, the substance may comprise combinations of biomolecules, for example the above-listed ribonucleoprotein (RNP) complexes, and the like.

The methods may further comprise subjecting a plurality of cells to flow through the channel and in contact with the compressive element. As provided herein, the compressive element may compress the cell or cells and facilitate the formation of one or more pores in cell membranes of at least a subset (e.g., at least about 10%, 15%, 20%, 25%, 30%, 35%, 40%, 45%, 50%, 55%, 60%, 65%, 70%, 75%, 80%, 85%, 90%, 95% of the cells, or more) of the cells. The one or more pores may permit at least a subset of the substances to enter or transport into the cell or cells to generate processed cells. The substances may be transported into the cell or cells with a high efficiency, for example, greater than or equal to about 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more. Additionally, the processed cells may have high cell viability. For example, after substance transportation or delivery, the processed cells may have cell viability that is greater than or equal to about 75%, 80%, 85%, 86%, 87%, 88%, 89%, 90%, 91%, 92%, 93%, 94%, 95%, 96%, 97%, 98%, 99%, or more.

As discussed above or elsewhere herein, the compressive element may comprise a plurality of compressive elements such as compressive surfaces. The plurality of compressive elements may be configured to conduct one or more compression-expansion cycles on the cell or cells. Expansion of the cell or cells after compression may be achieved by absorbing media surrounding the cell or cells. The surrounding media may comprise the substances. The substances may enter into the cell or cells via the one or more membrane pores formed during compression. The compressive elements may be a plurality of ridges in some examples. Dimensions of the compressive elements may be adjusted based on various factors, such as cell size, viscoelasticity, stiffness, or elasticity, and/or adhesion, flow rate, temperature, channel dimension, channel material, substance type, and/or substance size.

In some cases, to facilitate cell flow, at least a portion of an interior surface of the channel or a surface of the compressive element may be coated. The surface coating may be hydrophilic. The surface coating may be made from hydrophilic materials such as hydrophilic polymers.

Flow rate of the cells inside the channel may vary, depending upon specific applications. The flow rate may be constant or may vary along the channel as the cell or cells pass through the channel. The flow rate may be increased or decreased by altering dimensions of the channel and/or the compressive element(s). In some cases, the cell or cells may flow through the channel at an average rate of at least about 1 millimeter/second (mm/s), 5 mm/s, 10 mm/s, 20 mm/s, 40 mm/s, 60 mm/s, 80 mm/s, 100 mm/s, 150 mm/s, 200 mm/s, 250 mm/s, 300 mm/s, 350 mm/s, 450 mm/s, 500 mm/s, 550 mm/s, 600 mm/s, 650 mm/s, 700 mm/s, 750 mm/s, 800 mm/s, 850 mm/s, 900 mm/s, 1,000 mm/s, 1,200 mm/s, 1,400 mm/s, 1,600 mm/s, or even more. In some cases, the average flow rate is at most about 2,000 mm/s, 1,500 mm/s, 1,000 mm/s, 900 mm/s, 800 mm/s, 700 mm/s, 600 mm/s, 500 mm/s, 400 mm/s, 300 mm/s, 200 mm/s, 100 mm/s, 50 mm/s, or less. In some cases, the flow rate is between any of the two values described above, for example, between about 10 mm/s and about 750 mm/s or between about 10 mm/s and about 2000 mm/s.

Flow through the instant microfluidic devices can in some cases be described in terms of the rate of passage of fluid through the device. The fluid flow rate may also be increased or decreased by altering dimensions of the channel and/or the compressive element(s). In some embodiments, the fluid flows through the channel of the device at a rate of at least about 60 μL/min, 100 μL/min, 200 μL/min, 400 μL/min, 600 μL/min, 800 μL/min, 1000 μL/min, 1,200 μL/min, 1,400 μL/min, 1,600 μL/min, 1,800 μL/min, 2,000 μL/min, or even more.

In some cases, the cell or cells are suspended in a solution prior to being introduced into the microfluidic device. The solution may also comprise the substances, which substances may be mixed with the cells prior to being co-introduced into the microfluidic device. The solution may be a flow buffer. The solution may comprise one or more additional reagents. For example, the solution may comprise, in addition to the cell or cells and/or the substances, inhibitors of immune processes. In another example, the solution may comprise reagents that may modify one or more characteristics of the cell or cells, such as cell stiffness, elasticity and/or adhesiveness. Alternatively or additionally, the solution may comprise nanoparticles. The nanoparticles may comprise labels that may track the cells and/or substances. The nanoparticles may be nanoparticles trackers such as iron oxide nanoparticles.

In some cases, one or more sorting processes may be performed. The cell or cells may be sorted based on characteristics such as cell size, elasticity, stiffness, viscoelasticity, and/or adhesiveness.

Also provided in the present disclosure are methods and systems for intracellular delivery with high throughput. As a result, the methods and systems may be suitable for processing cells at a clinical scale. The methods and systems may include the use of a microfluidic device as described above or elsewhere herein. For example, the methods may comprise providing a microfluidic device which comprises a channel. The channel may comprise a compression element and a plurality of substances.

The methods may further comprise, flowing a cell or a plurality of cells through the channel during which the cell or cells may be in contact with the compression element. The cell or cells may pass through the channel at a high rate, e.g., a rate that is at least about 10⁷ cells/hour, 10⁸ cells/hour, 10⁹ cells/hour, 10¹⁰ cells/hour, or more. In some embodiments, the cell or cells pass through the channel at a rate of at least about 1×10⁸ cells/hour, 2×10⁸ cells/hour, 4×10⁸ cells/hour, 8×10⁸ cells/hour, or even more. As the cell or cells flow through the channel, the compression element may compress the cell or cells and facilitate the formation of at least one membrane pore in cell membrane of at least a subset of the cells. The at least one membrane pore may permit one or more substances to flow therethrough and enter into the cell or cells. In preferred embodiments, the at least one membrane pore is a transient pore. In particular, such transient pores close quickly after entry of the substance into the cell, so that the cell can quickly recover from the compression.

The microfluidic device may comprise a plurality of channels. As an example, the microfluidic device may comprise greater than or equal to about 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 70, 80, 90, 100 channels, or more. Each channel may comprise one or more compressive elements (e.g., greater than or equal to about 1, 2, 3, 4, 5, 6, 7, 8, 9, 10, 11, 12, 13, 14, 15 compressive elements, or more).

Individual channels of the plurality of channels may have the same or a different dimension. For example, the channels may have the same or a different cross-sectional dimension, length, width, and/or height. The channels may have the same or a different cross-sectional shape. The channels may be made from the same or a different material. The channels may or may not have surface coatings depending upon various factors such as cell type, size, stiffness, elasticity, viscoelasticity and/or stiffness. The channels may or may not be in fluidic communication with one another. In some cases, at least a subset (e.g., at least about 5%, 10%, 15%, 20%, or more) of the channels are in fluidic communication with one another. The plurality of channels may be arranged in parallel, in series or in a combined configuration of in parallel and in series. The plurality of channels may be in fluidic communication with a manifold. The cell or cells may be introduced into the microfluidic device comprising the channels via the manifold.

The cell or cells may be any type of cells as described above or elsewhere herein. The cell or cells introduced into the microfluidic device may comprise different types of cells. Each channel comprised in the microfluidic device may be configured to receive and process a different type of cells. The cell or cells may be processed with a high efficiency, e.g., greater than or equal to about 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more.

Within a given channel, in cases where multiple compressive elements are included, the compressive elements may have the same or a different dimension. For example, the compressive elements may have the same or a different height, width or length.

The compressive elements may be spaced apart from one another. A space between each pair of adjacent compressive elements may be the same or different. In some cases, the space between each pair of adjacent compressive elements may progressively increase or decrease along a flow direction of the cell or cells. The flow direction may be a principal axis of a given channel. The compressive elements may have a height that is smaller than a cross-sectional dimension of a channel within which the compressive elements are included. A gap may exist between each compressive element and an interior surface of the channel. Each compressive element may have the same or a different gap size (e.g., gap height). In some cases, at least a subset (e.g., greater than or equal to about 10%, 15%, 20%, 25%, 30%, 35%, 40%, 45%, 50%, or more) of the compressive elements have different gap sizes. The gap sizes may be determined based on various factors including, e.g., characteristics of the cell or cells such as cell size.

A ratio of the gap size to a cell size (e.g., cell diameter) may vary. In some cases, the ratio is greater than or equal to about 5%, 10%, 15%, 20%, 25%, 30%, 35%, 40%, 45%, 50%, 55%, 60%, 65%, 70%, 75%, 80%, or more. In some cases, the ratio may be less than or equal to about 90%, 80%, 70%, 60%, 50%, 40%, 30%, 20%, or less. In some cases, the ratio is between any of the two values described above, for example, between about 25% and about 75%, or between about 30% and about 60%.

The compressive elements may be parallel with respect to one another. The compressive elements may be angled relative to a principal axis of the channel within which the compressive elements are comprised. The angle may be an acute angle. In some cases, prior to introducing the cell or cells into the microfluidic device, the cell or cells may be sorted into different groups. The sorting may be based on cell type, size, shape, elasticity, stiffness, adhesiveness or combinations thereof.

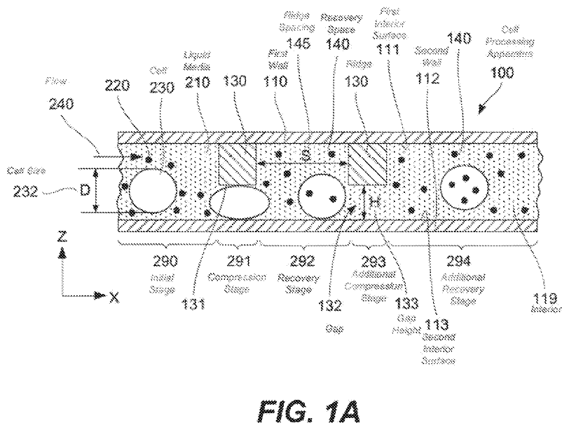

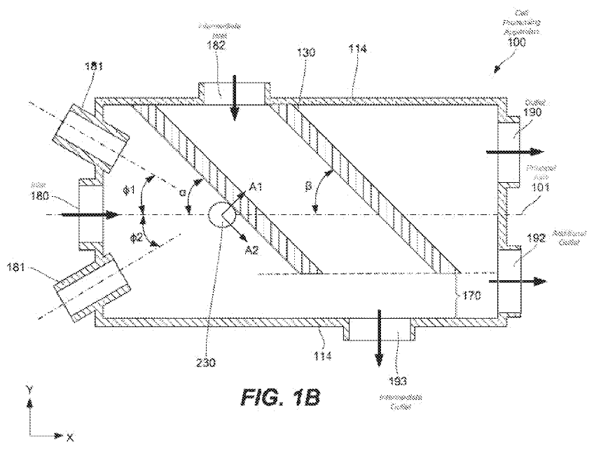

FIG. 1A is a schematic cross-section view of an example cell processing apparatus 100 for intracellular delivery, cell sorting, and/or other operations further described below. In some examples, cell processing apparatus 100 comprises first wall 110 and second wall 112. First wall 110 and second wall 112 may be also referred to as a top wall and a bottom wall, strictly for differentiation and without implying any orientation of cell processing apparatus 100. First wall 110 comprises first interior surface 111. In some examples, first interior surface 111 is planar. However, the interior surface may comprise other shapes. Likewise, second wall 112 comprises second interior surface 113, which may be also planar. In some examples, first interior surface 111 may be parallel to second interior surface 113. First interior surface 111 and second interior surface 113 may extend along the flow direction, identified with arrow 240 in FIG. 1A. First interior surface 111 and second interior surface 113 at least partially define interior 119 of cell processing apparatus 100. More specifically, first interior surface 111 and second interior surface 113 define the interior height (IH), which may impact the linear flowrate within interior 119. Interior 119 may be isolated from the environment and may be used to flow mixture 200, comprising liquid media 210, reagent 220, and cells 230.

In some examples, first wall 110 and/or second wall 112 may be formed from one or more transparent materials. For example, transparent materials of these walls may allow for integration of optical sensors into the cell processing apparatus 100 and/or other types of process control. On the other hand, nontransparent materials for the walls may be used to deliver light-sensitive reagents. Some examples of wall materials may comprise, but not be limited to, polydimethylsiloxane (PDMS), injection molded plastics, silicon, glass, and other polymers.

Referring to FIG. 1A, cell processing apparatus 100 may comprise a plurality of ridges 130, which may extend within interior 119 of cell processing apparatus 100. More specifically, in this example, plurality of ridges 130 may be connected to first wall 110 and extend within from first interior surface 111 and toward second interior surface 113. In some examples, cell processing apparatus 100 may comprises an additional plurality of ridges, which may be connected to the second wall 112 and extend within from second interior surface 113 and toward first interior surface 111. In some cases, the plurality of ridges 130 and the additional plurality of ridges may extend in the opposite direction and, in some examples, they may overlap along the height of the cell processing apparatus 100 (the Z-axis).

FIG. 1A illustrates two ridges forming plurality of ridge 130 extending from first wall 110. However, other numbers of ridges 130 can be used, such as, for example, one ridge, two ridges, three ridges, or four ridges. The number of ridges determines the number of compression cycles that some of cells 230 experience in a single pass through cell processing apparatus 100. Furthermore, additional compression cycles may be achieved by passing cells 230 through cell processing apparatus 100 multiple times.

Each of plurality of ridge 130 may comprises ridge surface 131, forming gap 132 with second interior surface 113. The height (H) of gap 132 may be smaller than the size/diameter (D) of cells 230, which may cause cells 230 to compress as cells 230 pass through gap 132. The compression may also depend on the flowrate and the length of ridge surface 131 (in the X direction), which may be also referred to as a ridge thickness. In some examples, the length of the ridge surface 131 and/or the ridge thickness may be between about 5 micrometers (μm) and 100 micrometers or, between about 20 micrometers and 50 micrometers. The length of the ridge surface 131 may be at least about 1 μm, 2 μm, 3 μm, 4 μm, 5 μm, 6 μm, 7 μm 8 μm, 9 μm, 10 μm, 12 μm, 15 μm, 20 μm, 25 μm, 30 μm, 35 μm, 40 μm, 45 μm, 50 μm, 55 μm, 60 μm, 65 μm, 70 μm, 80 μm, 90 μm, 100 μm, 110 μm, 120 μm, 150 μm, 200 μm, 300 μm, 400 μm, 500 μm, 1 millimeter (mm), or more. In some examples, the length of the ridge surface 131 may be at most about 1 mm, 800 μm, 700 μm, 600 μm, 500 μm, 400 μm, 300 μm, 200 μm, 150 μm, 100 μm, 80 μm, 70 μm, 60 μm, 50 μm, 40 μm, 30 μm, 20 μm, 15 μm, 10 μm, 5 μm, or less.

In some examples, all ridges (or a subset) of the plurality of ridges 130 may have the same length of ridge surface 131 and/or ridge thickness. Alternatively, the length of ridge surface 131 and/or ridge thickness may vary among the ridges. For example, upstream ridges (initial ridges along the flow direction) may have a shorter length of ridge surface 131 than downstream ridges. As such, the compression duration provided by these downstream ridges may be longer than that provided by the upstream ridges. The compression duration may also be impacted by the linear flow rates, which may be controllable by the cross-sectional areas of the cell processing apparatus 100, as further described below.

In some cases, when the length of ridge surface 131 is smaller than the cell size (D), the cell compressions can be compromised due to the cell ability to deform around the ridges, e.g., at least partially remain in uncompressed state when portions of the cell extend outside of gap 132. On the other hand, when the length of ridge surface 131 is much larger than the cell size, such as 10 times or more than the cell diameter, the cells may be prone to accumulation in gaps 132, which can lead to clogging.

Referring to FIG. 1A, in some examples, the cross-sectional profile (in a plane perpendicular to first interior surface 111 and second interior surface 113) of ridge 130 may be rectangular. However, other shapes of the profile are also within the scope, e.g., cylindrical, trapezoidal, or triangular. In some examples, the plurality of compressive surfaces may be orthogonal.

In some examples, ridge surface 131 may be parallel to the second interior surface 113. In other words, gap 132 may be defined by two parallel surfaces, one being ridge surface 131 and another one being a portion of second interior surface 113, and the gap thickness may be constant. Such parallel compressive surfaces may allow for a uniform compression for the entire cell. In some examples, the compression surfaces can be converging and/or diverging. Converging surfaces may allow for increasing the cell compression as the cells pass through the compressive space. Diverging surfaces can be used to allow cell expansion that accelerates cell motion and prevents clogging.

In some examples, the surface roughness of ridge surface 131 may be configured to increase cell membrane poration. For some materials, the surface roughness can be controlled using vapor etching. In some examples, the surface roughness with a mean size of between 10 nanometers (nm) and 1000 nm may be used. In some cases, the surface roughness may have a mean size of at least about 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 20 nm, 50 nm, 100 nm, 300 nm, 500 nm, 800 nm, 1000 nm, 1200 nm, nm, 1300 nm, 1500 nm, or more. In some cases, the surface roughness may have a mean size of less than or equal to about 2000 nm, 1500 nm, 1200 nm, 1000 nm, 800 nm, 600 nm, 500 nm, 400 nm, 300 nm, 200 nm, 100 nm, 50 nm, 20 nm, 10 nm, 5 nm, 1 nm, or less.

In some examples, the plurality of ridges 130 may be flexible (e.g., compliant). Flexible ridges may help to reduce cell damage. The ridge flexibility/compliance may be configured by selecting ridge material. In some examples, materials with modulus from 1 to 100 kPa may be used. Furthermore, ridge compliance may be configured using surface coatings with desired elasticity modulus.

Further referring to FIG. 1A, interior 119 may comprise recovery spaces 140, positioned between adjacent pair of plurality of ridge 130 and after the last ridge, along the flow direction/the X direction. In the Z direction, recovery spaces 140 may extend between first wall 110 and second wall 112. The height of recovery spaces 140 (in the Z direction between these walls) may be greater than the gap size. In some examples, the height of the recovery space 140 may be greater than the cell size (D). The height of recovery spaces 140 may be configured to allow the desired cell volume recovery, accompanied by cell expansion in the Z direction. The length of recovery spaces 140 (in the X direction) between two adjacent ridges may be referred to as ridge spacing 145, identified with the letter “S” in FIG. 1A. Ridge spacing 145 may determine the recovery duration, together with the linear flowrate. It has been found that volume gain (Vgain) may increase when the recovery time is increased. The recovery time can be increased by increasing ridge spacing 145. Other considerations for determining ridge spacing 145 may comprise cell characteristics, levels of previous compression, and the like. In some examples, ridge spacing 145 may be between 100 micrometers and 1000 micrometers such as between 200 micrometers and 500 micrometers.

Referring to FIG. 1B, cell processing apparatus 100 comprises side walls 114, comprising side interior surfaces 115. Side walls 114 may each be connected to each of first wall 110 and second wall 112, collectively forming interior 119. Side interior surfaces 115 may define the interior width (IW) of cell processing apparatus 100. Together with the interior height (IH), the interior width (IW) may impact the linear flowrate of mixture 200 through interior 119 or, more specifically, through recovery spaces 140. In some examples, the linear flowrate of mixture 200 as it passed through gaps 132 formed by plurality of ridge 130 may be much higher because of a much lower cross-sectional area corresponding to gaps 132 vs. recovery spaces 140 (the volumetric flowrate being the same).

Referring to FIG. 1B, cell processing apparatus 100 may comprise inlet 180 and outlet 190. In some examples, cell processing apparatus 100 may comprises one or more additional inlets 181. For example, multiple inlets may be used for supplying different cells and/or different reagents into cell processing apparatus 100. The inlets may be positioned at various angles relative to the flow direction, which in this example coincides with principal axis 101 of cell processing apparatus 100. For example, inlet 180 is shown to be parallel to the flow direction/principal axis 101. Additional inlets 181 are shown to be not parallel to the flow direction/principal axis 101 (e.g., ϕ1>0° and ϕ2>0°). The angle (ϕ1 and/or ϕ2) may be between 20° and 80° or, more in some examples, between 30° and 60°. In some examples, the angle (ϕ1 and/or ϕ2) may be greater than or equal to about 5°, 6°, 7° 8°, 9°, 10°, 12°, 15°, 20°, 25°, 30°, 35°, 40°, 50°, 55°, 60°, 65°, 70°, 75°, 80°, 85°, 90°, or more.

In some examples, inlet 180 may be a self-focusing inlet (e.g. with no sheath focus). The self-focusing inlet may use hydrodynamic focusing, such as Dean's flow effect. For example, inlet 180 may incorporate a focusing section, such as a serpentine channel, focusing ridges, focusing posts, focusing flow splitters, curved geometry using Dean's flow effect, inertial migration effect, and other methods leading to cross-stream cell migration. The focusing section may concentrate cells 230 at desired transverse location within the cell processing apparatus 100. Among other factors, the focusing location depends on the geometry of ridges 130 and ridge surface 131, which may be also referred to as compressive surfaces. For chevron ridges, the focusing location may be at the middle of the channel, in some examples. For diagonal ridges (e.g., shown in FIG. 1B), the focusing location may be biased to the side of diversion channel 170. Without a focusing section, a portion of cells 230 may be able flow from inlet 180 right into diversion channel 170, without being compressed by ridges 130, resulting in nonhomogeneous cell processing. In addition to focusing inlets, hydrodynamic flow may be directed by orientation of ridges 130 as further described below. Furthermore, in some examples, the hydrodynamic flow may be directed using electrical fields, such as electroosmotic flow, electrophoretic flow, and the like. Electrical, magnetic, thermal and other fields can be used to concentrate reagents 220 (e.g., macromolecules, nanoparticles) in specific locations within interior 119 of cell processing apparatus 100 to increase intracellular delivery into cells 230 as cells 230 may be compressed by ridges 130. For example, such effects electrophoresis, electroosmosis, thermophoresis, can be used to concentrate reagents near cells. Electrodes producing the fields can be integrated in walls of cell processing apparatus 100 and controlled by an external controller.

In some examples, a single inlet may be used to reduce an amount of reagents 220 that otherwise can be diluted by focusing a sheath fluid. At outlet 190, processed and unprocessed cells can be mixed for collection. An additional sorting device and operation can be used to separate unprocessed cells from mixture 200 after mixture 200 exists cell processing apparatus 100.

In some examples, cell processing apparatus 100 may comprise intermediate inlet 182, e.g., to introduce different reagents and reagent combinations for multistage cell processing. For example, intermediate inlet 182 may be used to introduce an additional mixture into recovery spaces 140 between adjacent ones of plurality of ridges 130. The composition of this additional mixture may be different from mixture 200, introduced upstream through inlet 180, which may be also referred to as a primary inlet.

In some examples, multiple outlets (e.g., outlet 190 and additional outlet 192) may be used for collecting different types of cells 230. As noted above, cell processing apparatus 100 may have cell sorting capabilities such that different types of cells 230 may flow into different portions of cell processing apparatus 100. Referring to FIG. 1B, less compressible cells may be directed by ridges 130 into diversion channel 170, while more compressible cells may pass through gaps created by ridges 130 and may stay away from diversion channel 170. Outlet 190 may be positioned away from diversion channel 170 and may be used for collecting cells 230 that have undergone compressions by ridges 130. Additional outlet 192 may be aligned with diversion channel 170 and may be used for collecting cells 230, which may be directed into diversion channel 170 and have not been compressed by desired number of ridges of plurality of ridges 130. In general, cell sorting characteristics, which determine whether cells 230 are directed into diversion channel 170 or undergo the compression include viscoelasticity, stiffness, or elasticity, and/or adhesion. Overall, multiple outlets may help to avoid clogging. Any number of outlets can be used one, two, three, four, or more.

In some examples, cell processing apparatus 100 may comprise intermediate outlet 193 as, for example, shown in FIG. 1B. For example, intermediate outlet 193 may be fluidically coupled to diversion channel 170 and open to diversion channel 170. Furthermore, intermediate outlet 193 may be disposed between a pair of plurality of ridges 130 as shown in FIG. 1B. Intermediate outlet 193 may be aligned with recovery space 140 between the pair of plurality of ridges 130. Intermediate outlet 193 may be used for collecting unwanted and abnormal cells and cell clusters, e.g., to prevent clogging of diversion channel 170 without passing these cells through the entire cell processing apparatus 100. In some examples, intermediate outlet 193 may be used to collect subpopulations of processed cells to improve delivery efficiency and uniformity.

Referring to FIG. 1B, all of plurality of ridges 130 may be diagonally-oriented relative to the general flow direction (shown with an arrow and coinciding with principal axis 101 of cell processing apparatus 100) within cell processing apparatus 100, i.e., from inlet 180 to outlet 190. In some cases, the smallest angle between ridges 130 and principal axis 101 may be an acute angle (α<90°). In some examples, the angle may be selected to provide hydrodynamic circulations in gaps 132 under ridges 130 (e.g., between ridge surface 131 and second interior surface 113). The angle of the ridges 130 can also affect the trajectories of cells 230 as, for example, schematically shown by directions A1 and A2 in FIG. 1B. The angle may depend on the flowrate, cell types, and other like parameters. In some examples, the angle may be between 10° to 80° or, more specifically, between 30° and 60°.

In some examples, all of plurality of ridges 130 may have the same angle relative to principal axis 101 (e.g., α=β, referring to FIG. 1B). In these examples, all ridges extend parallel to each other. Alternatively, some ridges in of plurality of ridges 130 may have different angles relative to principal axis 101 (e.g., α≠β) as, for example, is schematically shown in FIG. 1C. For example, a sharper angle may be used closer to inlet 180 (α<β) for early removal of abnormal cells and cell clusters in a less obstructive manner. A larger angle may be used further down the flow path (downstream) for faster cell compression and improved intracellular delivery. Principal axis 101 may be also referred to as the primary flow axis. It should be noted that while the flow may follow the principal axis 101, localized flow may vary, e.g., uncompressible cells may be diverted by a ridge to diversion channel 170.

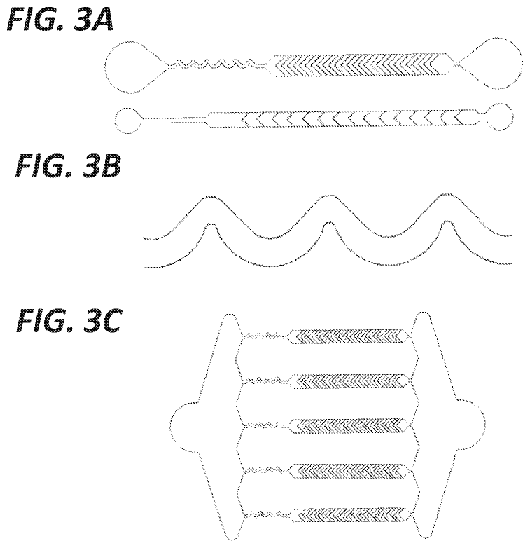

Referring to FIG. 1B, in some examples, ridges 130 may be in the form of straight bars, individually arranged in interior 119 of cell processing apparatus 100. In some examples, these straight bars may be arranged or even joined together into a chevron pattern as, for example, is shown in FIGS. 3A-3E. In this example, each of plurality of ridges may comprise a first ridge portion and a second ridge portion, having different orientations/positioned at different angles relative to the flow direction. It should be noted that the smallest angle between the flow direction and each of the first ridge portion and the second ridge portion may be the same. Alternatively, the smallest angle between the flow direction and each of the first ridge portion and the second ridge portion may be different. Furthermore, this smallest angle may be variable.

In some embodiments, the microfluidic devices of the instant disclosure may comprise multiple microchannels. (See, e.g., FIGS. 3C and 3D.) Such designs can substantially increase sample throughput. Alternatively or in addition, as shown in FIG. 3E, the width of the microchannels can be increased to increase sample throughput.

In some embodiments, the microchannels of the instant microfluidic devices may not include diversion channels. Although diversion channels can, in some device designs, advantageously provide a pathway for the passage of uncompressible cells (see, e.g., PCT International Application No. PCT/US19/64310, filed on Dec. 3, 2019), omission of diversion channels from a microfluidic device can, in other device designs, be desirable. Exemplary microchannel designs lacking diversion channels are shown in FIGS. 3A-3E. In particular, such designs can enable higher levels of intracellular delivery at higher flow rates for certain types of cells. In addition, such designs can be manufactured using a wider variety of methods and materials than devices comprising microchannels with diversion channels. For example, these designs can be readily prepared using standard methods of injection molding. See, e.g., Example 3 below for evidence demonstrating the benefit of devices lacking diversion channels.

In some embodiments, the first wall and second wall of the disclosed microfluidic devices are substantially rigid walls. The instant inventors have discovered that previous device designs, where a least one wall of the device is composed of a relatively flexible material, for example polydimethylsiloxane (PDMS), the wall can be distorted by the pressure of fluid flowing through the microchannels of the device, particularly as flow rates are increased. Such distortion can increase the spacing between the first wall and the second wall of the device, thus increasing the gap height or heights 133 (see FIG. 1A). An increased gap height can result in lower and/or less consistent transfection efficiency as cells pass through the device, in particular as fluid flow rates are increased. See, e.g., Example 3 below.

The efficiency of intracellular delivery of substances to cells according to the instant methods and devices may, in some embodiments, be expressed in terms of transfection efficiency. For example, where the substance being delivered to the cell is a nucleic acid, for example double-stranded DNA or a messenger RNA, transfection efficiency in the instant methods and devices can be at least 20%, 30%, 40%, 50%, 60%, 70%, 80%, 90%, or even higher.

The efficiency of processing may, in other embodiments, be expressed in terms of product yield or total transfection, where the value is calculated by multiplying transfection efficiency and recovery of live cells, in order to understand the percentage of cells engineered from the total number of cells input in the system or the overall throughput. In some embodiments, the product yield obtained using the instant methods and devices can be at least 10%, 20%, 30%, 40%, 50%, 60%, 70%, 80%, 90%, or even higher.

In some embodiments, performance of the instant methods and devices is assessed by the viability of cells that have been processed using the methods and devices of the instant disclosure. In some embodiments, the viability of cells is at least 10%, 20%, 30%, 40%, 50%, 60%, 70%, 80%, 90%, or even higher. Alternatively or in addition, performance of the methods and devices is assessed by the recovery of cells from the process. In some embodiments, the recovery of cells is at least 10%, 20%, 30%, 40%, 50%, 60%, 70%, 80%, 90%, or even higher.

Additional exemplary methods and systems for intracellular delivery are provided in PCT International Application No. PCT/US19/64310, filed on Dec. 3, 2019, the disclosure of which is incorporated herein by reference in its entirety.

### Delivery of Substances to Vertebrate Blood Cells

The methods and systems of the present disclosure can advantageously be used in the delivery of substances to vertebrate blood cells, in particular vertebrate white blood cells or leukocytic cells. Examples of such cells include monocytes, basophils, eosinophils, neutrophils, and lymphocytes. Of particular interest is the delivery of substances to lymphocytes, for example B cells, T cells, natural killer cells (NK cells), natural killer T cells (NKT cells), gamma delta T cells (γδ T cells), and the like.

T cells, in general, are of particular interest, in view of their recent use in novel immunotherapeutic agents and methods, such as T cells that have been engineered to express an artificial T cell receptor for use in targeted immunotherapies. For example, chimeric antigen cell receptors (CARs) comprising both an antigen binding function and a T cell activating function within a single chimeric protein, have recently been expressed in transfected T cells (i.e., CAR-T cells). When the antigen binding function of the chimeric receptor directs the transfected CAR-T cell to a tumor-associated antigen, ideally a tumor-specific antigen, the cell can become activated, can proliferate, and can ultimately become cytotoxic towards the targeted tumor cell.

Although the methods and systems of the present disclosure are preferably used to deliver a nucleic acid to a target cell, it should be understood that any suitable substance can be delivered to any suitable cell under appropriate conditions. In some embodiments, the substance is more generally a biopolymer, such as, for example, a nucleic acid, a polysaccharide, a polypeptide, or any combination of these biopolymers. In other embodiments, the substance is a small-molecule drug. In some embodiments, the substance is an antigen, including protein antigens and haptens. In some embodiments, the substance is a charged substance.

In some embodiments, the nucleic acid substance is a deoxyribonucleic acid (DNA) or a ribonucleic acid (RNA). In some embodiments, the nucleic acid substance is a nucleic acid analog, for example a peptide nucleic acid (PNA), a morpholino or locked nucleic acid (LNA), a glycol nucleic acid (GNA), a threose nucleic acid (TNA), or any combination thereof. In some embodiments, the nucleic acid substance is a messenger RNA (mRNA) or a transfer RNA (tRNA).

In some embodiments, the nucleic acid substance encodes an RNA or protein activity of interest. For example, the nucleic acid substance may encode a fluorescent or luminescent protein, such as a green fluorescent protein (GFP) or a luciferase, the nucleic acid substance may encode a gene editing protein, such as a CRISPR-associated protein, for example Cas9, dCas, Cas9 RNP, dCas RNP, or any combination of these proteins.

In some embodiments, the nucleic acid substance is a viral nucleic acid, including a viral DNA or a viral mRNA.

In some embodiments, the polypeptide substance is an antibody or an enzyme.

In some embodiments, the substance is a gene-editing reagent, such as a CRISPR-associated protein, for example Cas9, dCas, Cas9 RNP, dCas RNP, or any combination of these proteins.

### Use of Different Sizes of Subsets of Immune Cells to Tune T Cell Transfection.

The instant inventors have discovered that the biophysical properties of particular T cells before and after activation can be exploited to optimize transfection using microfluidic devices of the instant disclosure.

In particular, it is known that T cell subsets display biophysical differences. See, e.g., Rossi et al. (2019) Lab Chip 19(22):3888-3898 (https://doi.org/10.1039/C9LC00695H). The average values for each biophysical property for CD4+ cells from all the donors were compared with values for the same properties of CD8+ cells. For unstimulated cells: dimension of CD4+ is 7.180±0.109 m, dimension of CD8+ is 7.214±0.175 m; the nuclear to cytoplasm ratio (n/c ratio) of CD4+ is 0.954±0.005, while n/c ratio of CD8+ is 0.968±0.003. Values of standard deviation between all donors never exceeded 5% of the corresponding average value. It is important to highlight that a single biophysical property for the cells is not sufficient to identify CD4+ or CD8+, both in the case of unstimulated cells and after IL-15 stimulation. The percentage of overlapping area between the data range confirms the consolidated knowledge that T-lymphocyte subclasses are extremely similar from a morphological point of view.

Accordingly, in some embodiments, the conditions for transfection of T cells using the instant devices can be optimized to take advantage the biophysical differences between CD4+ and CD8+ cells, in particular, differences size, shape, elasticity, stiffness, adhesiveness or combinations thereof. For example, cells can be sorted according to one or more biophysical property using an initial microfluidic device, and the sorted cells can be transfected using a second device (or a second microchannel in the same device), where the conditions for transfection in the second device (e.g., gap size) have been tuned to the optimal conditions for those cells. Multiple ridge structures can be incorporated such that each structure is tuned to transfect desired subsets of T cells, for example by changing the ridge spacing.

### Use of Formulations to Enhance Plasmid Delivery.

Formulations can be designed to compact DNA and mitigate charge, as well improve the active transport to the nucleus. To enhance compaction the following reagents can be considered.

Poly(ethylene glycol)-block-poly(propylene glycol)-block-poly(ethylene
glycol), average Mn ~4,400
Poly(ethylene glycol)-block-poly(propylene glycol)-block-poly(ethylene
glycol), average Mn ~2,000
Pluronic ® F-127, powder, BioReagent, suitable for cell culture
Poloxamer 188
15-Crown-5, 98%
Polyvinylpyrrolidone, average mol wt 360,000
Polyvinylpyrrolidone, average mol wt 10,000
Polyvinylpyrrolidone, average mol wt 40,000
Poly(ethylene glycol), BioUltra, 1,000
Poly(ethylene glycol), BioUltra, 2,000
Poly(ethylene glycol), BioUltra, 4,000
Poly(ethylene glycol), BioUltra, 8,000
Poly-L-glutamic acid sodium salt, mol wt 3,000-15,000
Poly-L-glutamic acid sodium salt, mol wt 1,500-5,500 by MALLS
Poly-L-glutamic acid sodium salt, mol wt 15,000-50,000

The use of RNase-free media may be important to maintain nucleic acid stability.

Also Pluronic-block copolymers can be used to passivate cargo, cells, or device surfaces. Pluronic polymers are composed of an internal polyoxypropylene (hydrophobic) chain bordered by external polyoxyethylene (hydrophilic) chains, have been previously shown to have some use in gene therapy. The variation of species within this group is determined by two factors: the ratio of hydrophobic to hydrophilic chains and the total molecular weight of the species.

Calcium phosphate can be added to the buffer to improve transfections. It is key to make the CaP particles not grow too big, which can be controlled with salt concentration as described by prior literature (https://www.ncbi.nlm.nih.gov/pmc/articles/PMC3647690/).

### Use of Formulations to Enhance Nuclear Transport of Plasmid.

### Active Transport to Nucleus

Including plasmid sequences with a SV40 enhancer sequence and CMV promoter can be designed to improve plasmid transport. Also Importin-B interacting proteins can be included in the formulation. Transcription factors bound to the DNA interact with importin β and other proteins that link the complex to dynein and kinesin for movement along microtubules toward the nucleus. Nuclear entry is then mediated by importin β in a sequence- and importin-dependent manner through the nuclear pore complex (NPC) in non-dividing cells or independent of importins and any DNA sequence requirement during mitosis and the associated dissolution of the nuclear envelope. Additional proteins in the trafficking DNA complex included the nuclear localization signal receptor proteins importin β1, importin 4, importin 7, importin α1, and importin α2, as well as numerous DNA-binding proteins and chaperones. Also CREB binding sites can be included by incubating naked DNA with cell extracts or recombinant importin molecules. DNA can also be biotinylated DNA and include avidin-NLS (nuclear localizing sequences). Non-NLS pathways, glyco-dependent nuclear import is thought to mediate the nuclear translocation of glycosylated plasmids. Nuclear transport can also be accomplished by increasing the functional diameter of the NPC itself by enhancing non-selective gating of the pore with the drug TCHD. The nuclear transport can also occurs through induced mechanical damage, but is undesirable from cell health standpoint.

The use of the viral package to help deliver DNA is possible, for example de-activated virus or capsid-less packages. Another method is through the incorporation of the particles into the nucleus during cell division. DNA binding proteins (DBPs) are capable of binding DNA and have been exploited as DNA carriers in gene delivery.

### Modified Cells

In another aspect, the disclosure provides cells that have been modified according to any of the above-described methods using any of the above-described microfluidic devices. As already described, cells modified using traditional ex vivo modification methods are often irreversibly changed or damaged as a result of the process. For example, cells transfected using chemical or viral agents typically contain residual chemical or viral components for at least some time after the treatment. In some cases the residual components can remain within the modified cells permanently.

In the case of electroporation, transfection efficiency and cell viability can be low, thus limiting the yields of modified cells achievable using the method. In addition, off-target variations in gene expression can occur, thus indicating alterations in nuclear and other cellular components that arise as a result of the electroporation process. Electroporated cells can also be slow to recover proliferative capacity, further indicating undesirable alterations in the chemical and biological functions of the modified cells.

In contrast, modified cells obtained using the instant methods and devices suffer fewer modifications or other negative consequences as a result of the process than cells obtained using other traditional intracellular delivery techniques. For example, cells modified using the instant methods or devices recover quickly from the treatments. Without intending to be bound by theory, it is understood that cells can rapidly recover from a compressed state by absorbing surrounding media through one or more transient pores in their cellular membranes. After the cells have expanded and recovered some or all of the volume lost during the compression, the one or more pores are no longer present in the membranes, and the cells recover the ability to proliferate.

In some embodiments, a cell modified according to the instant methods or devices proliferates within 10 days of delivery of a substance into the cell. More specifically, the cell proliferates within 8 days, 7 days, 6 days, 5 days, 4 days, 3 days, 2 days, 1 day, or even sooner. In other more specific embodiments, the cell proliferates within 48 hours of delivery of a substance into the cell. More specifically, the cell proliferates within 36 hours, 24 hours, 18 hours, 12 hours, 6 hours, 3 hours, or even sooner.

In some embodiments, a cell modified according to the instant methods or devices is substantially free of a transfection agent. More specifically, the cell is substantially free of a chemical transfection agent or a biological transfection agent.

In some embodiments, a T cell modified according to the instant methods or devices retains high proliferative capacity and/or cytotoxic potential. In some embodiments, a T cell modified according to the instant methods or devices displays low levels of exhaustion markers. In some embodiments, a CD34+ cell modified according to the instant methods or devices retains high proliferative capacity.

In any of the above embodiments, the product yield of modified cells can be at least 20%, 30%, 40%, 50%, 60%, 70%, 80%, 90%, or even higher.

### Alternative Methods

In another aspect, the disclosure provides methods for delivering at least a subset of a plurality of substances into at least a subset of a plurality of cells, as described in the following numbered paragraphs.

1. A method for delivering at least a subset of a plurality of substances into at least a subset of a plurality of cells, comprising:
(a) providing a microfluidic device comprising a channel comprising a compressive element and said plurality of substances, which said plurality of substances has an average molecular weight greater than or equal to about 1 megadaltons (MDa); and
(b) subjecting said plurality of cells to flow through said channel and in contact with said compressive element, wherein said compressive element facilities formation of one or more pores in cell membranes of said at least said subset of said plurality of cells, which one or more pores are sufficient to permit said at least said subset of said plurality of substances to enter said at least said subset of said plurality of cells at an efficiency greater than or equal to about 50%.
2. The method of paragraph 1, wherein said average molecular weight is greater than or equal to about 2 MDa.
3. The method of paragraph 1, wherein each of said plurality of substances has a molecular weight greater than or equal to about 1 MDa.
4. The method of paragraph 1, wherein said efficiency is greater than or equal to about 90%.
5. The method of paragraph 1, wherein said one or more pores are transient pores.
6. The method of paragraph 1, wherein said channel has a cross-sectional dimension between about 20 micrometers (μm) and about 1,000 μm.
7. The method of paragraph 6, wherein said cross-sectional dimension is between about 50 μm and about 100 μm.
8. The method of paragraph 1, further comprising, forming a coating on at least a portion of an interior surface of said channel.
9. The method of paragraph 8, wherein said coating is hydrophilic.
10. The method of paragraph 9, wherein said coating comprises hydrophilic polymers.
11. The method of paragraph 1, wherein a gap between said compressive element and an interior surface of said channel is between about 3 μm and about 15 μm.
12. The method of paragraph 1, wherein a gap between said compressive element and an interior surface of said channel is less than or equal to about 20% of an average diameter of said plurality of cells.
13. The method of paragraph 1, wherein a gap between said compressive element and an interior surface of said channel is less than or equal to about 20% of a diameter of a given cell of said plurality of cells.
14. The method of paragraph 1, wherein said compressive element comprises ridges.
15. The method of paragraph 14, wherein said ridges extend parallel with respect to one another.
16. The method of paragraph 14, wherein said ridges have rectangular cross-sections.
17. The method of paragraph 14, wherein said ridges have an average width between 20 micrometers (μm) and 250 μm.
18. The method of paragraph 14, further comprising, forming a coating on at least part of surfaces of said ridges.
19. The method of paragraph 1, wherein said plurality of cells flows through said channel at a flow velocity from 10 millimeter/second (mm/s) to 750 mm/s.
20. The method of paragraph 1, wherein said compressive element comprises a plurality of compressive surfaces.
21. The method of paragraph 20, wherein a space between each pair of adjacent compressive surfaces progressively increases along a flow direction of said plurality of cells.
22. The method of paragraph 1, wherein said compressive element is configured to deform said at least of said subset of said plurality of cells, thereby creating said one or more pores.
23. The method of paragraph 1, wherein said compressive element is configured to reduce a volume of said at least of said subset of said plurality of cells.
24. The method of paragraph 23, wherein said volume is reduced temporarily.
25. The method of paragraph 1, wherein said plurality of substances comprises a charged substance.
26. The method of paragraph 1, wherein said plurality of substances comprises a drug, a nucleic acid molecule, an antigen, a polypeptide, an antibody, an antigen, a hapten, an enzyme, or combinations thereof.
27. The method of paragraph 26, wherein said nucleic acid molecule comprises deoxyribonucleic acid (DNA), ribonucleic acid (RNA), peptide nucleic acid (PNA), or combinations thereof.
28. The method of paragraph 1, wherein said plurality of cells comprises T cells, hematopoietic stem cells (HSCs), induced pluripotent stem cells (iPSCs), Chinese hamster ovary (CHO) cells, or combinations thereof.
29. The method of paragraph 1, further comprising, prior to (a), suspending said plurality of cells in a fluid comprising said plurality of substances.
30. The method of paragraph 29, wherein said fluid is a flow buffer.
31. The method of paragraph 30, further comprising, adding reagents to said flow buffer.
32. The method of paragraph 31, wherein said reagents comprise inhibitors of immune processes.
33. The method of paragraph 31, wherein said reagents comprise reagents that modify stiffness and/or elasticity of said plurality of cells.
34. The method of paragraph 31, wherein said reagents comprise nanoparticle trackers.
35. The method of paragraph 34, wherein said nanoparticle trackers comprise iron oxide nanoparticles.
36. The method of paragraph 1, further comprising, prior to (a), sorting said plurality of cells based on cell size, viscoelasticity, stiffness and/or adhesiveness.
37. The method of paragraph 1, further comprising, modifying said plurality of substances using nuclear locators.
38. The method of paragraph 37, wherein said nuclear locators comprise nuclear localization signal (NLS) tags.
39. A method for delivering at least a subset of a plurality of substances into at least a subset of a plurality of cells, comprising:
(a) providing a microfluidic device comprising a channel comprising a compressive element and said plurality of substances; and
(b) subjecting said plurality of cells to flow through said channel and in contact with said compressive element, wherein said compressive element facilities formation of one or more pores in cell membranes of said at least said subset of said plurality of cells, which one or more pores are sufficient to permit said at least said subset of said plurality of substances to enter said at least said subset of said plurality of cells to generate processed cells at an efficiency greater than or equal to about 50%, wherein each of said processed cells has a cell viability greater than or equal to about 85%.
40. The method of paragraph 39, wherein said efficiency is greater than or equal to about 80%.
41. The method of paragraph 39, wherein said efficiency is greater than or equal to about 90%.
42. The method of paragraph 39, wherein said cell viability is greater than or equal to about 95%.
43. The method of paragraph 39, wherein said compressive element comprises a plurality of compressive surfaces.
44. The method of paragraph 43, wherein a space between each pair of adjacent compressive surfaces progressively increases along a flow direction of said plurality of cells.
45. The method of paragraph 39, wherein said compressive element is configured to deform said at least said subset of said plurality of cells, thereby creating said one or more pores in said cell membranes.
46. The method of paragraph 39, wherein said compressive element is configured to reduce a volume of said at least of said subset of said plurality of cells.
47. The method of paragraph 46, wherein said volume is reduced temporarily.
48. The method of paragraph 39, wherein said compressive element comprises ridges.
49. The method of paragraph 48, wherein said ridges extend parallel with respect to one another.
50. The method of paragraph 48, wherein said ridges have rectangular cross-sections.
51. The method of paragraph 39, wherein said plurality of cells flows through said channel at a flow velocity from 10 millimeter/second (mm/s) to 750 mm/s.
52. The method of paragraph 39, wherein said plurality of substances comprises a charged substance.
53. The method of paragraph 39, wherein said plurality of substances comprises a drug, a nucleic acid molecule, an antigen, a polypeptide, an antibody, an antigen, a hapten, an enzyme, or combinations thereof.
54. The method of paragraph 53, wherein said nucleic acid molecule comprises deoxyribonucleic acid (DNA), ribonucleic acid (RNA), peptide nucleic acid (PNA), or combinations thereof.
55. The method of paragraph 39, wherein said plurality of substances comprises gene-editing reagents.
56. The method of paragraph 39, wherein said plurality of substances comprises green fluorescent protein (GFP) DNA plasmid, GFP mRNA, Cas9, dCas, Cas9 RNP, dCas RNP, or combinations thereof.
57. The method of paragraph 39, wherein said plurality of cells comprises T cells, hematopoietic stem cells (HSCs), induced pluripotent stem cells (iPSCs), Chinese hamster ovary (CHO) cells, or combinations thereof.
58. The method of paragraph 39, further comprising, prior to (a), suspending said plurality of cells in a fluid comprising said plurality of substances.
59. The method of paragraph 58, wherein said fluid is a flow buffer.
60. The method of paragraph 59, further comprising, adding reagents to said flow buffer.
61. The method of paragraph 60, wherein said reagents comprise inhibitors of immune processes.
62. The method of paragraph 60, wherein said reagents comprise reagents that modify stiffness and/or elasticity of said plurality of cells.
63. The method of paragraph 60, wherein said reagents comprise nanoparticle trackers.
64. The method of paragraph 63, wherein said nanoparticle trackers comprise iron oxide nanoparticles.
65. The method of paragraph 39, further comprising, prior to (a), sorting said plurality of cells based on cell size, viscoelasticity, stiffness and/or adhesiveness.
66. A method for delivering at least a subset of a plurality of substances into at least a subset of a plurality of cells, comprising:
(a) providing a microfluidic device comprising a channel comprising a compressive element and said plurality of substances; and
(b) subjecting said plurality of cells to flow through said channel and in contact with said compressive element at a rate greater than or equal to about 10⁸ cells/hour, wherein said compressive element facilities formation of one or more pores in cell membranes of said at least said subset of said plurality of cells, which one or more pores are sufficient to permit said at least said subset of said plurality of substances to enter said at least said subset of said plurality of cells.
67. The method of paragraph 66, wherein said microfluidic device comprises a plurality of channels.
68. The method of paragraph 67, wherein said microfluidic device comprises at least 5 channels.
69. The method of paragraph 67, wherein said microfluidic device comprises at least 10 channels.
70. The method of paragraph 67, wherein each of said plurality of channels comprise one or more compressive elements.
71. The method of paragraph 67, wherein each of said plurality of channels has the same cross-sectional dimension.
72. The method of paragraph 67, wherein individual channels of said plurality of channels have different cross-sectional dimensions.
73. The method of paragraph 67, wherein said plurality of channels is in fluidic communication with a manifold.
74. The method of paragraph 67, wherein said plurality of cells is introduced into said microfluidic device via said manifold.
75. The method of paragraph 67, wherein said plurality of cells comprises different types of cells.
76. The method of paragraph 75, further comprising, directing said different types of cells into said microfluidic device.
77. The method of paragraph 76, further comprising, introducing each type of said cells into a different channel.
78. The method of paragraph 66, wherein said rate is greater than or equal to about 10⁹ cells/hour.
79. The method of paragraph 66, wherein said one or more pores are sufficient to permit said at least said subset of said plurality of substances to enter said at least said subset of said plurality of cells at an efficiency greater than or equal to about 50%.
80. The method of paragraph 66, wherein said compressive element comprises a plurality of compressive surfaces.
81. The method of paragraph 80, wherein a space between each pair of adjacent compressive surfaces progressively increases along a flow direction of said plurality of cells.
82. The method of paragraph 66, wherein said compressive element comprises ridges.
83. The method of paragraph 82, wherein said ridges extend parallel with respect to one another.
84. The method of paragraph 82, wherein said ridges have rectangular cross-sections.
85. The method of paragraph 82, wherein said ridges are angled relative to a principal axis of said channel.
86. The method of paragraph 85, wherein said angle is less than about 90°.
87. The method of paragraph 66, wherein said plurality of cells flows through said channel at a flow velocity from 10 millimeter/second (mm/s) to 750 mm/s.
88. The method of paragraph 66, wherein said plurality of substances comprises a charged substance.
89. The method of paragraph 66, wherein said plurality of substances comprises a drug, a nucleic acid molecule, an antigen, a polypeptide, an antibody, an antigen, a hapten, an enzyme, or combinations thereof.
90. The method of paragraph 89, wherein said nucleic acid molecule comprises deoxyribonucleic acid (DNA), ribonucleic acid (RNA), peptide nucleic acid (PNA), or combinations thereof.
91. The method of paragraph 66, wherein said plurality of substances comprises gene-editing reagents.
92. The method of paragraph 66, wherein said plurality of substances comprises green fluorescent protein (GFP) DNA plasmid, GFP mRNA, Cas9, dCas, Cas9 RNP, dCas RNP, or combinations thereof.
93. The method of paragraph 66, wherein said plurality of cells comprises T cells, hematopoietic stem cells (HSCs), induced pluripotent stem cells (iPSCs), Chinese hamster ovary (CHO) cells, or combinations thereof.
94. The method of paragraph 66, further comprising, prior to (a), suspending said plurality of cells in a fluid comprising said plurality of substances.
95. The method of paragraph 94, wherein said fluid is a flow buffer.
96. The method of paragraph 66, further comprising, adding reagents to said flow buffer.
97. The method of paragraph 96, wherein said reagents comprise inhibitors of immune processes.
98. The method of paragraph 96, wherein said reagents comprise reagents that modify stiffness and/or elasticity of said plurality of cells.
99. The method of paragraph 96, wherein said reagents comprise nanoparticle trackers.
100. The method of paragraph 99, wherein said nanoparticle trackers comprise iron oxide nanoparticles.

It should be understood throughout the disclosure that whenever the term “at least,” “greater than,” or “greater than or equal to” precedes the first numerical value in a series of two or more numerical values, the term “at least” or “greater than” applies to each one of the numerical values in that series of numerical values.

In addition, it should be understood that whenever the term “no more than,” “less than,” or “less than or equal to” precedes the first numerical value in a series of two or more numerical values, the term “no more than” or “less than” applies to each one of the numerical values in that series of numerical values.

The term “about” or “nearly” as used herein generally refers to within +/−15%, 10%, 9%, 8%, 7%, 6%, 5%, 4%, 3%, 2%, or 1% of the designated value.

As used herein, the singular forms “a”, “an”, and “the” include plural references unless the context clearly dictates otherwise.

It will be readily apparent to one of ordinary skill in the relevant arts that other suitable modifications and adaptations to the methods and applications described herein can be made without departing from the scope of the invention or any embodiment thereof. Having now described the present invention in detail, the same will be more clearly understood by reference to the following Examples, which are included herewith for purposes of illustration only and are not intended to be limiting of the invention.

### EXAMPLES

### Example 1. Use of Microfluidic Devices to Deliver Nucleic Acids to Cells

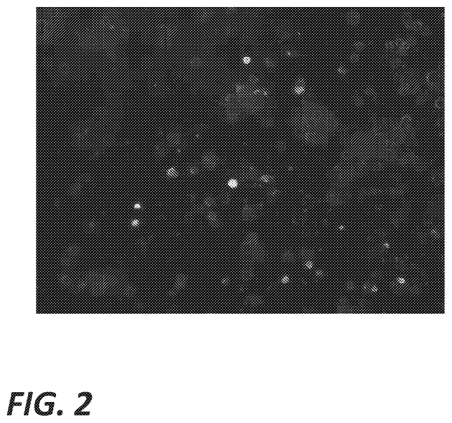

Plasmid delivery to cells: K562 cells are subjected to flow through a microfluidic device of the present disclosure. The microfluidic device comprises a channel comprising one or more compressive elements and plasmids. As the cells pass through the channel and in contact with the compressive elements, the cells are experiencing one or more compression-expansion cycles during which the plasmid molecules are actively transported into the cells. The experiment successfully induces EGFP expression after 1 day of culture with delivery of EGFP plasmid to K562 cells (FIG. 2). A single microfluidic channel may process at least 4×10⁶ cells per minute. The microfluidic device may be comprised of multiple channels which may increase this amount substantially (see FIGS. 3C and 3D).

Microfluidic mechanoporation: Using methods and systems of the present disclosure, molecules in the size up to 2 MDa are delivered into cells with high efficiency. The method maintains high cell viability (>80% when measured with both acridine orange and propidium iodide stain or flow cytometry) compared to unprocessed control cells. The processing rate is over 4×10⁶ cells/min for extended time without clogging. Cell volume was found to be temporarily changed as measured using high-speed video microscopy. However, the cells rapidly returned to their normal size when compared to unprocessed control cells and wild type control cells. Expression was not observed to change for apoptotic and cytoskeletal markers.

Cell processing of primary T cells: Ex vivo genetic engineering of T cells holds great promise as a route to durable and complete elimination of cancer by reprogramming the patient's own immune system to attack the cancer. In CAR-T cell therapy, a chimeric antigen receptor (CAR) arms a CD4+/CD8+ T cell to hunt and direct the killing of all cells expressing a target on their surface. In TCR T cell therapy, a T cell receptor (TCR) targeting a specific cancer neoantigen may be similarly employed. Using methods and systems from the present disclosure, CRISPR/Cas9 system can be delivered to knock-out TCR function with a cell viability >80% and efficiency >50% when measured with flow cytometry after 5 days of expansion.

Various challenges remain for engineered T cells in cancer, many of which are related to manufacturing. To date CAR T may have been made through retroviral gene transfer, followed by cell expansion and formulation. Because the virus inserts into the host genome at random, it may carry an intrinsic risk of genotoxicity. Manufacture of high-quality virus under GMP conditions at sufficient quantity can be time-consuming and expensive, and lot-to-lot variability (both of virus drug substance and of resulting CAR T cell drug product) may lead to manufacturing failures.

Alternative methods may comprise electroporation coupled with genome editing to insert the CAR (or TCR) at a specified “safe harbor” locus. However, electroporation can be difficult to scale, and often results in lower knock-in efficiency than viral transduction. Additionally, the above-mentioned may have limits on the size of the transgene delivered, whether through limitations of viral packaging (˜8 kb for lentiviral gene transfer) or through limitations of reagent diffusion to the cell interior (for electroporation and other passive diffusion approaches).

Intracellular delivery of T cells using the methods and systems of the present disclosure have shown successful delivery to primary cells of therapeutic interest, while preserving viability and expansion potential. Primary T cells purchased from healthy donors (both fresh and frozen). Cells are cultured with T-cell cytokine, IL-2, and treated with T-cell Activation agent, TransACT (commercially available) and washed with DPBS then resuspended 24 hours later in medium containing payload. These cells are processed using the methods and systems of the present disclosure to evaluate the impact of cell processing on long-term cell viability and expansion. After cells are processed and cultured for 1-5 days (dependent on payload) the cells were evaluated using flow cytometry to identify CD4+ and CD8+ T-cell populations. The cells maintain viability >80% when compared to unprocessed control cells, >70% transfection efficiency, and the capacity to expand under culture.

Microfluidic mechanoporation of iPS cells: Induced pluripotent stem (iPS) cells are processed to deliver large vectors and express large genes (>8 kilobase pairs) using microfluidic delivery of the present disclosure. The gene includes a GFP reporter with multiple Crispr constructs.

Cell processing to deliver GFP plasmids, GFP mRNA, and GFP transposon (knock-in) to CD34+ HSPC: Microfluidic devices are made with polydimethylsiloxane (PDMS) replica molding using SU-8 photoresist patterned onto a silicon substrate through standard microfabrication techniques. Glass braces are inserted into the PDMS mold before curing to increase channel integrity. After curing of PDMS, inlet and outlet capillaries are added and each device is sealed against a glass slide substrate. Dead volume of microfluidic channel and regions are reduced without compressions and focusing sheath flows are eliminated by using Deans focusing channels as in FIGS. 3A-3C.

HSPC mechanoporation and analysis: Primary CD34+ hematopoietic stem cells (HSPC) are purchased from commercial sources. Cells are thawed and cultured in serum-free medium with HSPC cytokines (Flt-3L, SCF, TPO) before resuspension in culture medium containing GFP plasmid payload. A reporter gene plasmid is used. For comparison, electroporation is used as controls. 24 hours after treatment, GFP expression is assessed by flow cytometry, along with flow cytometry for human CD34 (to assess purity) and human CD38 (to assess engraftment potential and stem-ness), with results compared to control transfection and untreated cells. The results show total cell viability >75% and transfection (or delivery) efficiency >40% post-cell processing, as assessed by flow cytometry.

Gene editing with CRISPR/Cas9 (RNP) in HSPC: Cas9 RNPs that target the first exon of human CD55 are designed. Guide RNA synthetically as a single guide with 3 3′ and 3 5′ protection (3xMS-sgRNA), along with purified wild-type Cas9 protein are purchased from commercial sources. Final selection of guide RNA is assessed using either electroporation or microfluidic treatment of K562 cells and a T7 endonuclease assay. Guide “3XMS-G10” is used to edit adult β-globin (HBB). To edit HSPC using devices of the present disclosure, Cas9 RNP is firstly assembled in water. The RNP is then mixed with CD34+ HSPC in cell culture medium, which mixture is subsequently directed to flow through the device. HSPC is sampled 1 day after treatment to assess viability, purity, and stem-ness by flow cytometry for CD34 and CD38 Treated HSPC is expanded for a minimum of 5 days before assessment of CD55 knockout by flow cytometry for CD55. After gene editing, the total cell viability is >80% with an efficiency >40%.

### Example 2. Manufacture and Operation of a Microfluidic Device for the Delivery of Substances to Cells

Exemplary microchannel designs for intracellular delivery are described in PCT International Application No. PCT/US19/64310, filed on Dec. 4, 2019, and in references cited therein. Additional microchannel designs are provided in FIGS. 3A-3E. Microfluidic devices including such microchannel designs can be prepared quickly, simply, inexpensively, and reliably from polydimethylsiloxane (PDMS), which is an organosilicone material capable of solidifying in the presence of a crosslinker and moderate heat. The material enables a high volume of devices to be manufactured for purposes of design optimization and testing.

VECT devices come in a variety of gap sizes. These gap sizes are correlated with a pre-produced silicon wafer. The wafer provides several devices per PDMS manufacturing run and each wafer can be used indefinitely if properly maintained. The resulting devices display certain gap sizes, and each gap size can be used by anyone trained in performing R&D and optimization testing of certain cell types. Those involved in manufacturing PDMS should also have knowledge of maintaining the silicon wafer templates.

Genetic modification can, for example, be performed on peripheral blood mononuclear cells (PBMCs) using the above-described devices with the addition of a suitable substance or substances, for example a plasmid, an mRNA, and/or a CRISPR/Cas9 system. While each respective payload may result in a somewhat different outcome, performance of mechanoporation on PBMCs is similar for most kinds of payloads. Alterations of the following protocol can be performed in order to improve and/or optimize the recovery, viability, or transfection rate as well as scaling of the device.

### Buffer Formulations:

1. Device buffer
   a. TexMacs media 1.5 ml/channel tested
   b. Superase RNAse inhibitor 1:1000
   c. 15% nuclease free water if performing CRISPR/CAS9 experimentation
2. Complete culture medium
   a. TexMacs media 0.6 ml/channel tested
   b. 1:100 PS
   c. 1:1000 IL-2
3. Nuclease-free DPBS
   a. 1.0 ml 10× nuclease-free DPBS
   b. 9.0 ml nuclease-free water

### Sterilization and Preparation of Working Environment:

1. Prior to experimentation ensure that all tubing elements (inlet syringe tip and assembled outlet tubing) have been autoclaved and remain sterile in a container
2. Wipe hood, syringe pump, and lab-jack with RNAse ZAP wipes
3. Place pump, lab-jack, and devices in TC hood
4. Clean autoclaved tubing and syringe tip container with RNAse ZAP wipes. Additionally, clean pipettes, and nuclease-free pipette tips before putting in TC hood
5. Once setup add 500 ul of complete culture media to wells of a 24-well plate. Aliquot as many wells as the number of channels being used, plus additional wells for ND controls. Place in 37 C, 5% CO₂ incubator to equilibrate media

### Passivation of Devices:

1. Add 10-15 ml of RNAse ZAP and nuclease-free water to separate, labeled 50 ml conicals
2. Add sterile syringe tip to sterile individually packaged 3 ml syringe. Aspirate 3 ml of RNAse ZAP from the conical into the syringe. Removing any air bubbles from syringe as necessary
   a. This can be done by withdrawing 1 ml, inverting syringe, tapping syringe until bubbles are at top of liquid front, and slowly pushing the plunger down. Once liquid starts coming out of the syringe tip the remaining volume can be taken out of the conical
3. Once loaded place the syringe on the syringe pump. Lock syringe into place and put hammer down on the back of the syringe plunger gently.
4. Set syringe pump parameters, flow rate: 800 ul/min, flow volume: 1.0 ml, syringe diameter 8.66 mm
5. Attach outlet tubings to the device outlets that will be used.
6. Firmly attach the syringe tip to the inlet of the device
   a. Ensure the syringe tip is not contacting the glass portion of the device as this will result in very high pressures
7. Run syringe pump flowing through 1 ml of RNAse ZAP
   a. Syringes can be duplexed to run two at a time
   b. If more than three channels need to passivated remove device, add more RNAse ZAP to syringe and passivate the next channels
8. Once passivating devices with RNAse ZAP passivate devices with 1 ml of nuclease-free water using same process
9. After passivation with water is complete repeat the process using VECT buffer. This can be performed with lower flow volume for each channel: 0.5 ml
   Treat Cell Samples with Payload:
1. Count cells in culture. Record culture density and viability. Identify how many cells are needed to obtain a final concentration of 2.0E+6 cells/ml in final payload mixture.
   a. If culture density is low this concentration can be adjusted
   b. PBMCs can be tested in naïve state or 24 hours after activation¹
2. Add required number of cells to 15 ml conical
3. Spin samples at 300×g for 5 minutes. Aspirate media
4. Wash cells with 1 ml of nuclease-free DPBS, resuspend cells using p1000 pipette.
   a. Samples can be transferred to nuclease-free centrifuge tubes at this point
      i. Final payload volume if transferring to centrifuge tubes will be 1.0 ml
5. Spin samples at 300×g for 5 minutes. Aspirate DPBS
6. Add relevant payload to the cells (when thawing new vial of plasmid/mRNA/sgRNA, aliquot and add information to traceability documentation)
   a. Plasmid
      i. Add plasmid to a concentration of 100 ug/ml to the cell pellet. Mix via pipetting
   b. mRNA
      i. Add mRNA to a concentration of 150 ug/ml to the cell pellet. Mix via pipetting
   c. CRISPR/Cas9 (adjust amounts as necessary for testing (Cas9:sgRNA ratio is 1:1)²
      i. Immediately after the first spin of the cells aliquot 4 ul of Cas9 protein (stock—5 ug/ul) to a nuclease free tube
      ii. Add 100 ul of TexMacs media to Cas9 protein
   iii. Add 1.56 ul of relevant sgRNA (stock—100 pmol/ul)
   iv. Incubate mixture at RT for 15 minutes
   v. After washing cells add 100 ul of Cas9 complex (Cas9 RNP) to cell pellet. Mix via pipetting
7. Add a necessary amount of VECT buffer to the top of payload to ensure concentrations of cells and genetic payloads are correct
8. Mix payloads by gently tapping bottom of tubes

### Running Cells and Payload Through VECT Device:

1. Add sterile syringe tip to 1 ml sterile syringe
2. Withdraw 0.8 ml of payload from centrifuge tube into syringe removing air bubbles like the aforementioned method
3. Setup syringe pump so it is vertical on the lab-jack with. Place the syringe point down on the syringe pump
4. Set syringe pump parameters, flow rate: 800 ul/min, flow volume: 0.28 ml, syringe diameter 4.78 mm
5. Attach device to syringe tip and run samples through
6. As the syringe pump flows discard the first 0.08 ml of the sample and capture only the last 0.2 ml in a centrifuge tube
7. Make sure to capture 0.2 ml of a ND sample. This is a control of cells treated with payload but not put through device
8. Once all channels are run and samples collected the samples can be resuspended
   a. For mRNA or Plasmid add 200 ul of each sample directly to 500 ul of complete cell media that was placed in the incubator
   b. For Cas9 RNP, wash samples spinning at 300×g for 5 minutes, aspirated, and resuspending with 200 ul of equilibrated media. Add resuspended samples to 96-well plater
9. Incubate samples at 37 C, 5% CO₂ 24-96 hours before analysis
   a. 24 for mRNA and plasmid
   b. 96 for Cas9 RNP

### Example 3. Microfluidic Devices Designed for Higher Sample Throughput and Higher Transfection Rates

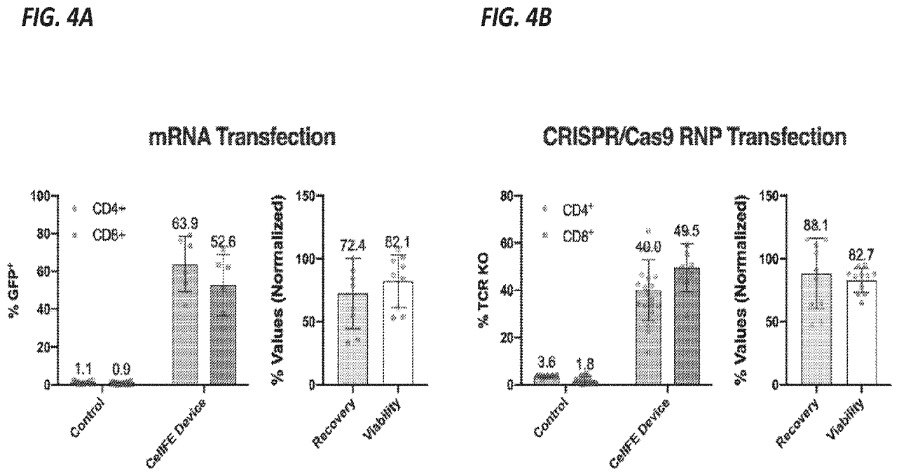

FIGS. 4A-4E illustrate the deformed channel geometry that can occur in a microfluidic device with non-rigid channel walls as the rates of fluid flow through the channel are increased. In this device, the top channel wall is manufactured from PDMS by replica molding as described above. The device is viewed in cross-section from the side. The interface between the top channel wall and the fluid within the channel is highlighted by the upper dotted line in each cross-section. The bottom channel wall is a glass slide to which the PDMS mold is fused. The interface between the bottom channel wall and the fluid within the channel is highlighted by the lower dotted line in each cross-section. A single ridge is visible in the middle of each micrograph as the darker cross section extending downward from the top channel wall. The device shown in FIGS. 4A-4E was designed with an 8 μm gap between the ridge and the bottom channel wall.

As the flow rate of fluid through the channel is increased from 0 to 400 μL/min, the PDMS top channel wall flexes upward, thus increasing the gap between the ridge and the bottom channel wall from 8 μm (in FIG. 4A) to 16-18 μm (in FIG. 4E). The upward flex is highlighted in FIG. 4D by the small arrows. As the instant inventors have discovered, this flexing can be significantly minimized by designing a microfluidic device with substantially rigid channel walls. For example, and as shown in FIG. 4F, by including a glass brace backing across the region of PDMS where the ridge is located, the increase in gap size between the ridge and the bottom channel wall can be minimized as the flow rate through the device increases. Alternatively, the device can be manufactured using a substantially rigid material to define the surfaces of the channel, for example by injection molding of the channel surfaces with a thermoplastic or a thermosetting polymer that results in a device with substantially rigid channel walls. For purposes of the disclosure, it should be understood that the channel walls of a microfluidic device are considered to be substantially rigid if the gap between at least one ridge in the device and the bottom channel wall does not increase significantly under fluid flow rates used to process samples within the device.

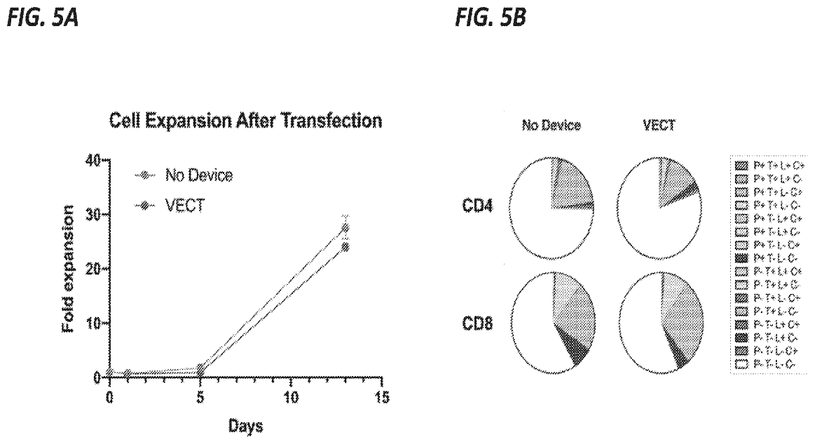

FIGS. 5A-5B show various views around the last ridge in another device with glass brace backing to support the PDMS channel surface in the region of the ridge. As shown in FIG. 5A, in the absence of fluid flow, the measured channel height in this device was 25.8 μm, the measured ridge height was 21.6 μm, and the measured gap size was 4.25 μm. As shown in FIG. 5B, even with a fluid flow of 800 μL/min, there was little or no change in the measured gap size in this device.

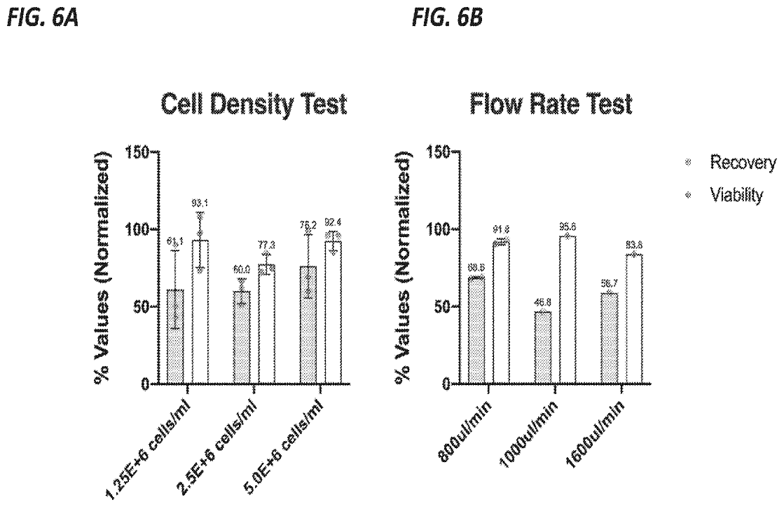

As shown in FIGS. 6A and 6B, the use of higher flow rates in a device with channel walls that are substantially rigid causes somewhat reduced viability and recovery of cells that are processed through the device. As shown in FIG. 6C, the transfection rates of CD4+ and CD8+ cells is increased significantly at the higher flow, and as shown in FIG. 6D, the total transfected cell yield remains relatively constant as the flow rate is increased. This result indicates that an overall higher throughput can be achieved with these devices with no loss to total product yield.

Even higher cell throughput can be achieved using wider channels, for example as illustrated in the novel channel designs of FIG. 3E.

As described above, the microchannels of the instant microfluidic devices may in some cases be designed with no diversion channels, for example as shown in the microchannel designs of FIGS. 3A-3E. The omission of diversion channels has been shown in some of these designs to improve transfection rates of peripheral blood mononuclear cells, including T cells. For example, FIGS. 7A-7D summarize transfection results for CD4+ and CD8+ T cells using various device designs. The results compare devices with either 4.2 μm gaps (dark shading) or 4.9 μm gaps (light shading). The devices compared in these experiments contained 12 chevron ridges, either with (“12 Ridge”) or without (“12 Ridge—NG”) a diversion channel, 5 chevron ridges in the middle of the microchannel flow path, either with (“5 Ridge (Middle)”) or without (“5 Ridge (Middle)—NG”) a diversion channel, and 5 chevron ridges at the end of the microchannel flow path, either with (“5 Ridge (Back)”) or without (“5 Ridge (Middle)—NG”) a diversion channel. As shown in these experiments, transfection of both CD4+ and CD8+ T cells increased significantly in most microchannel designs when the diversion channels were omitted.

### Example 4. Transfection of Peripheral Blood Mononuclear Cells with CRISPR/Cas9 and mRNA

CRISPR/Cas9 has been used to knock out the T cell receptor (TCR) functionality in PBMCs from two donors. As shown in FIG. 8A, the knockout efficiency is 34% and 52% for CD4+ cells and CD8+ cells, respectively, and the viability and recovery of cells is high using the VECT device (labeled as “CellFE Device”).

FIG. 8B shows a flow cytometry analysis of untreated (top) or VECT-treated (bottom) cells. TCR knockout cells are present at high levels in the VECT-treated samples for CD4+ (left) and CD8+ (right) cells, respectively.

PBMCs have also been transfected with mRNA using a microfluidic device of the instant disclosure (“CellFE Device”). As shown in FIG. 9, the levels of transfection of CD4+ and CD8+ cells were high, and the viability and recovery of cells were high with the VECT-treated samples. Data were from 2 different PBMC donors. mRNA concentration was 45 μg/mL.

### Example 5. Comparison of Transfection of Unactivated and Activated T Cells with mRNA

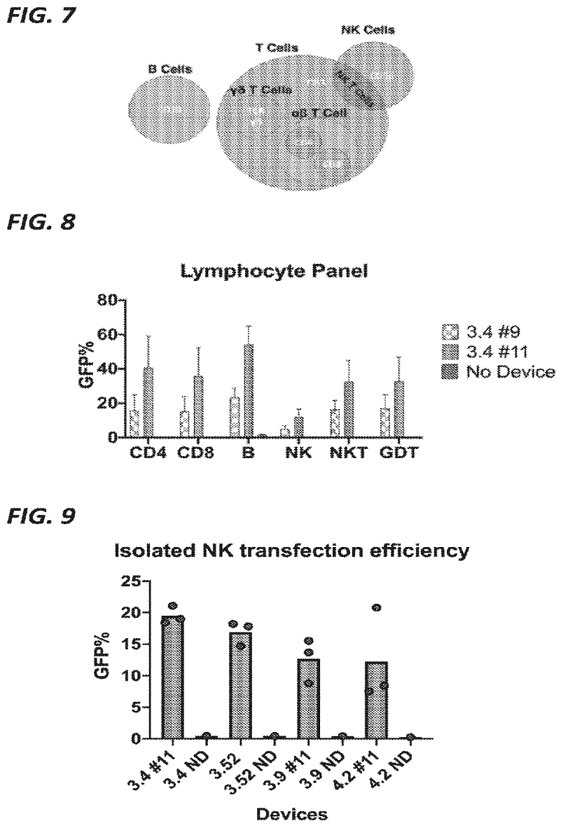

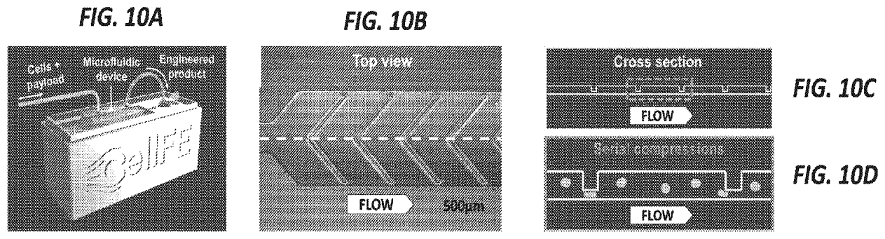

FIG. 10 shows a comparison of mRNA transfections using two different VECT devices with naïve T cells and the corresponding transfection of activated T cells. In all cases, high levels of transfection were achieved, together with high levels of cell viability and recovery.

### Example 6. Novel Microfluidic Platform for Scalable Transfection of mRNA and CRISPR/Cas9 RNP in Human T Cells

Genetically engineered human T-cells present a promising platform to advance treatments of refractory cancers and solid tumors. Currently, the manufacturing of genetically engineered T-cells relies heavily on the production of costly, hard to scale-up lentiviral vectors. On the other hand, the most prominent non-viral alternative for the transfection of T cells is electroporation, encumbered by cell loss and disruption of normal cell function. A novel microfluidic based platform that relies on the process of volume exchange for convective transfer (VECT) has been developed to transfect cells in a high-throughput manner.

Fresh PBMCs were cultured and activated before transfection. Before VECT, cells were washed and placed in fresh native media together with payload: GFP mRNA at 45 μg/ml, or TRAC CRISPR/Cas9 RNP at 18 μg/ml. Cells were then collected and placed back in culture. Transfection efficiency was studied at 24 h (mRNA), or more than 5 days (RNP) after VECT. For cell expansion results, cells were processed using VECT and mRNA, and cultured in G-rex for two weeks until all readouts were collected. Device capacity was tested using GFP mRNA.

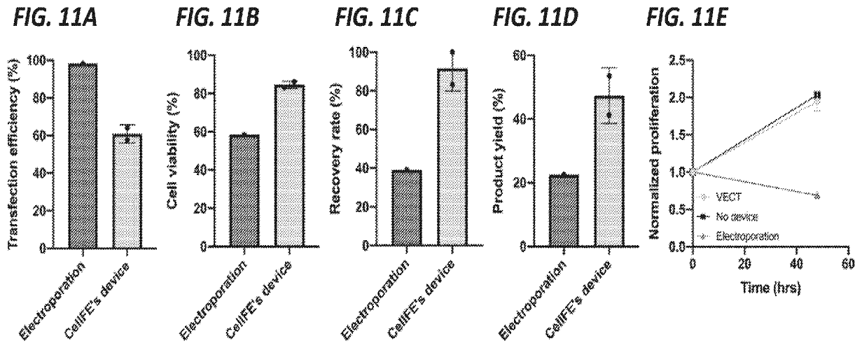

As shown in FIGS. 11A and 11B, VECT results in high transfection efficiencies of both mRNA (>60% and >50%) and RNP molecules (>40% and >50%) into CD4+ and CD8+ T cells, respectively. Viability of the cells is high (>80%), whereas high recovery might be partially influenced by the payload of choice (>70% in mRNA vs >80% in RNP).

T cell expansion upon VECT was approx. 24-fold in a period of 13 days after transfection (FIG. 12A). VECT did not promote exhaustion in processed cells (FIG. 12B).

Parameters to enable processing of higher number of cells were assessed. The device can successfully operate at high density of cells (up to 5 million cells/ml) (FIG. 13A) and high flow rates (up to 1600 μl/min) (FIG. 13B). Running the device continuously, it can process 50 million cells in less than 7 minutes. Cell viability and recovery were comparable within each variable tested; mRNA transfection efficiency was also similar.

The VECT transfection device enables high transfection of T cells with both mRNA and CRISPR/Cas9 RNP payloads, while preserving viability and overall cell number recovered from the device. VECT does not promote exhausted T cell phenotypes. Finally, processing parameters have been validated that will enable the manufacturing of 50 million cells processed in under 10 minutes employing a single channel device.

### Example 7. Microfluidic-Enabled Delivery of mRNA in Human NK and Gamma Delta T Cells

Natural Killer (NK) cells and Gamma Delta (γδ) T are lymphocytes that demonstrates to be promising therapeutic cell carriers because they can be used in allogeneic CAR treatments. Contrasting to autologous CAR-T immunotherapy, with the allogeneic approach cells can be pooled from healthy donors to produce a cost effective “off-the-shelf product”. It can be administered to multiple patients in a more readily accessible manner. The current methods to generate oncology gene therapies have shown to be less than ideal for NK-cells immunotherapy. A microfluidic device has been developed that can induce transient volume exchange in cells, resulting in cell transfection with payloads of interest. Shown here is the successful transfection of nucleic acids (i.e. mRNA) into NK cells and γδ T cells.

Naive PBMCs flowed through devices with varying gap sizes at a uniform flow rate (μl/min) and mRNA concentration (μg/ml). Later, NK cells were first isolated and activated before flowing through the devices.

Readouts were taken via a flow cytometer 24 hours after the initial experiment. A lymphocyte panel was designed to enable a quick screen of various lymphocyte populations including two cell types of interest: NK cells and γδ T cell, as shown in FIG. 14. Expression of GFP in the transfected cells is illustrated in FIGS. 15 and 16. As shown in these figures, mRNA was successfully transfected in NK cells and γδ T cells. Transfection efficiency differed from donor to donor, averaging at about 20% for NKs and 30% for γδ T with the best devices.

### Example 8. Microfluidic Device for Intracellular Delivery of Nucleic Acids into Human CD34+ Hematopoietic Stem and Progenitor Cells

Human CD34+ hematopoietic and progenitor cells (HSPCs) constitute a keystone cell carrier in the advent of ex vivo gene therapy. Challenges remain in the genetic engineering process of these cells, preventing HSPC gene therapy products from becoming more inexpensive and accessible to patients.

Volume exchange for convective transfer (VECT) offers a microfluidic-based, non-viral, scalable alternative to the genetic engineering of CD34+ cells. Transfection by VECT is achieved when cells are flowed at high speed through a series of subcellular compressions (FIGS. 17A-17D), resulting in an active transport of matter from the surrounding media into the cell. In VECT, cells are flowed in their native media mixed with a payload of interest.

This example illustrates the use of a novel microfluidic device for the transfection of nucleic acids (e.g. mRNA) into human HSPC cells, and a comparison of those results with results obtained in a commercially available electroporator.

CD34+ cells were thawed and cultured under standard conditions for 48 hours, before being mixed with GFP mRNA payload and 1) electroporated following a commercial protocol, 2) transfected by flowing through an exemplary VECT device, or 3) returned unprocessed to cell culture as a negative control (i.e. no device control). All readouts were measured 48 h after transfection.

Although the VECT device shows lower transfection efficiency than the electroporator system (FIG. 18A), CD34+ cell viability and recovery rate are greater in VECT than in electroporation (FIGS. 18B and 18C). When these variables are considered together, the product yield of the VECT device is approximately double that of electroporation (FIG. 18D). Finally, it is also shown that CD34+ cells are capable of proliferating successfully after VECT, growing at a rate equivalent to the negative control (FIG. 18E), while electroporated CD34+ cells tend to display lower proliferation in the first 48 h of culture after transfection.

VECT produces double the yield of transfected cells than that of commercial electroporation. Moreover, VECT transfected cells can readily proliferate at a rate comparable to the negative control, illustrating that the treated cells maintain better cell function than electroporation. Taken together, the results show that VECT constitutes a novel, competitive platform for HSPC transfection.

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

1. A method for delivering a substance into a cell, comprising:
(a) providing a microfluidic device, wherein the microfluidic device comprises a channel that comprises a compressive element; and a fluid within the microfluidic device, wherein the fluid comprises the cell and the substance; and
(b) subjecting the fluid to flow through the channel in contact with the compressive element, wherein the contact causes formation of at least one pore in a membrane of the cell, wherein the at least one pore enables an entry of the substance into the cell.

2. The method of claim 1, wherein the entry of the substance into the cell is at an efficiency greater than or equal to about 50%.

3. The method of claim 1, wherein the substance has an average molecular weight greater than or equal to about 1 megadaltons.

4. The method of claim 1, wherein the cell is a vertebrate blood cell.

5-10. (canceled)

11. The method of claim 1, wherein the substance is a nucleic acid.

12-14. (canceled)

15. The method of claim 1, wherein the substance is a gene editing reagent.

16. (canceled)

17. The method of claim 1, wherein a gap between the compressive element and an interior surface of the channel is between about 3 μm and about 15 μm.

18. The method of claim 1, wherein the cell has a cell diameter, and wherein a gap between the compressive element and an interior surface of the channel is less than or equal to about 20% of the cell diameter.

19. The method of claim 1, wherein the compressive element is a ridge.

20. (canceled)

21. The method of claim 1, wherein the cell flows through the channel at an average flow rate of from 10 mm/s to 2000 mm/s.

22-24. (canceled)

25. The method of claim 1, wherein the fluid comprises a population of cells, and wherein the substance enters at least 50% of the population of cells.

26-27. (canceled)

28. The method of claim 1, wherein the fluid further comprises a nanoparticle tracker.

29. (canceled)

30. The method of claim 1, wherein the method further comprises the step of selecting the cell for a biophysical property prior to subjecting the fluid to flow through the channel in contact with the compressive element.

31. The method of claim 30, wherein the biophysical property distinguishes CD4+ cells from CD8+ cells.

32. The method of claim 30, wherein the biophysical property is size.

33. The method of claim 30, wherein the biophysical property is presence of a specific surface antigen.

34. The method of claim 1, wherein the channel is defined by at least a first wall and a second wall, wherein the first wall and the second wall are substantially rigid.

35. The method of claim 34, wherein the channel does not comprise a diversion channel.

36. The method of claim 34, wherein the first wall comprises a flexible material and a bracing material, and wherein the bracing material is positioned on an exterior surface of the first wall.

37. (canceled)

38. The method of claim 34, wherein the first wall or the second wall is prepared by injection molding.

39-76. (canceled)
